(19) United States
(12) Patent Application Publication
(10) Pub. No.: US 2013/0288317 A1
(43) Pub. Date: Oct. 31, 2013

(54) INCREASED YIELDS OF PHAS FROM HYDROGEN FEEDING AND DIVERSE CARBON FIXATION PATHWAYS

(71) Applicant: Metabolix, Inc. Chemical, Cambridge, MA (US)

(72) Inventors: Thomas M. Ramseier, Cambridge, MA (US); Christopher W.J. McChalicher, Cambridge, MA (US)

(21) Appl. No.: 13/830,296

(22) Filed: Mar. 14, 2013

Related U.S. Application Data
(60) Provisional application No. 61/643,048, filed on May 4, 2012.
(60) Provisional application No. 61/640,679, filed on Apr. 30, 2012.

(51) Int. Cl. C12P 7/62; C12P 7/18

(52) U.S. Cl. CPC C12P 7/625; C12P 7/18
USPC 435/135; 435/252.3; 435/158; 435/254.11; 435/257.2; 435/252.33; 435/252.34; 435/252.31; 435/252.2; 435/252.32; 435/254.21; 435/254.2; 435/254.3; 435/254.23

(57) ABSTRACT

Disclosed are methods including organisms genetically engineered to make useful products when grown on glucose as a carbon source. The organisms are genetically engineered to produce various useful products such as polyhydroxyalkanoates (PHA) monomers, polymers, and copolymers, diols, alcohols, and other useful chemicals.

### RELATED APPLICATIONS

This application is a continuation-in-part and claims the benefit of U.S. Provisional Application No. 61/643,048, filed on May 4, 2012 and U.S. Provisional Application No. 61/640,679, filed on Apr. 30, 2012. The entire teachings of the above applications are incorporated herein by reference.

### INCORPORATION BY REFERENCE OF MATERIAL IN ASCII TEXT FILE

This application incorporates by reference the Sequence Listing contained in the following ASCII text file:

a) File name: 46141009002SEQUENCELISTING.txt; created Mar. 14, 2013, 4 KB in size.

### BACKGROUND OF THE INVENTION

Polyhydroxyalkanoates (PHA's) polyesters are naturally produced in many types of bacterial cells as a form of intracellular carbon and energy storage. The most common PHA type produced by bacterial cells is poly-3-hydroxybutyrate (P3HB) which was first identified in Bacillus megaterium by Lemoigne in 1926. Consumer products utilizing PHA's are being commercially developed as an alternative to products made from petroleum-based polymers due to their excellent material performance properties and their ability to biodegrade in diverse environments such as soil, marine and home compost at the end of their product life. To support the commercial growth of PHA's as a versatile polymer material, genetically-modified biomass systems have recently been developed which produce a wide variety of biodegradable PHA polymers and copolymers in high yield (Lee (1996), Biotechnology & Bioengineering, 49: 1-14; Braunegg et al., (1998), J. Biotechnology, 65:127-161; Madison and Huisman (1999), Metabolic Engineering of Poly-3-Hydroxyalkanoates; From DNA to Plastic, in Microbiol. Mol. Biol. Rev., 63:21-53). The PHA's produced from genetically-modified biomass systems are typically prepared via a fermentation process utilizing feedstocks such as sugars, fatty acids, alcohols or vegetable oils as sources of carbon for the PHA production. After fermentation, the polymer is isolated, dried and processed into the desired products.

One of the objectives in the development of microbial strains for PHA production is to maximize the yield from the fermentation process where the yield is defined as weight PHA/weight carbon source fed. The yields of these processes are often however limited by metabolic pathways for recycling carbon lost in the form of carbon dioxide and inefficient balancing of electrons. A route to mitigate these carbon losses in PHA producing strains would be incorporating genes from microorganisms that are capable of utilizing carbon dioxide and hydrogen to make the PHA.

Organisms from the phylogenic branch called Archaea have the ability to utilize hydrogen and carbon dioxide and are often found living in extreme temperature or caustic environments. Although archaea are visually similar to bacteria (both are prokaryotic organisms), they were classified in the 1970s as actually belonging to a third distinct phylogenic branch. Archaea possess genes and several metabolic pathways that are more closely related to those of eukaryotes, notably the enzymes involved in transcription and translation. Other aspects of archaeal biochemistry are unique, such as their reliance on ether lipids in their cell membranes. Archaea also use a much greater variety of sources of energy than eukaryotes: ranging from familiar organic compounds such as sugars, to ammonia, metal ions or even hydrogen gas. Salt-tolerant archaea (the Haloarchaea) use sunlight as an energy source and other species of archaea fix carbon; however, unlike plants and cyanobacteria, no species of archaea is known to do both.

Several different autotrophic carbon fixation pathways have been identified in archaea organisms (Berg (2010), Microbiology, vol. 8, June, p 447) which when incorporated in genetically-modified bacterial strains can be used to significantly increase the overall product yields. The most important metabolic pathways are those that biosynthesize chemical intermediates which can then be used to produce products of interest. Thus a need exists for combining energy pathways in an organism to increase yields of desired products.

### SUMMARY OF THE INVENTION

Described herein are methods of utilizing at least one enzyme of a carbon fixation pathway in combination with polyhydroxyalkanoate (PHA) pathways for increases of product yields. The methods include organisms that have been modified to incorporate a carbon fixation pathway, a PHA pathway or both pathways and optionally hydrogen and carbon dioxide co-feeds. Increased yields of certain intermediates and end products from the incorporated metabolic pathways in these resultant organisms are achieved by the methods of the invention. While aspects of these pathways are unique, certain intermediates and enzymes are shared and manipulation and incorporation of genes that encode enzymes of these pathways allows the generation of increased yields of products.

In a first aspect of the invention, methods of increasing a yield of a polyhydroxyalkanoate (PHA) monomer, polymer or co-polymer in an organism utilizing a carbon feedstock, for example, glucose (or other sugars such as sucrose, sugars derived from cellulosic hydrolysates and the like) as a carbon source and having a PHA pathway, comprising incorporating in the organism a carbon fixation pathway for increasing levels of the (PHA) monomers, polymers or co-polymers compared to the organism before incorporation of the carbon fixation pathway are described.

In a second aspect of the invention, methods of increasing a yield of polyhydroxyalkanoate (PHA) monomer, polymer or co-polymer in an organism utilizing glucose (or other sugars such as sucrose, sugars derived from cellulosic hydrolysates and the like) as a carbon source, comprising incorporating in the organism a carbon fixation pathway and a PHA pathway, and producing a polyhydroxyalkanoate (PHA) monomer, polymer or co-polymer with increased yield over the organism before incorporation of the pathways are described.

In a third aspect, methods of increasing a yield of a polyhydroxyalkanoate (PHA) monomer, polymer or co-polymer in an organism utilizing glucose (or other sugars such as sucrose, sugars derived from cellulosic hydrolysates and the like) as a carbon source and having a carbon fixation pathway comprising incorporating in the organism a PHA pathway pathway, wherein the yield of the (PHA) monomers, polymers or co-polymers compared to the organism before incorporation of the carbon fixation pathway are described.

In a first embodiment, of any of the aspects of the invention, the carbon fixation pathway is a 3HP/4HB cycle pathway or a 3HP bi-cycle pathway.

In a second embodiment of invention, the monomer is 3-hydroxypropionate, 3-hydroxybutyrate, 4-hydroxybutyrate, or 5-hydroxyvalerate, the polymer is poly-3-hydroxypropionate, poly-3-hydroxybutyrate, poly-4-hydroxybutyrate, or poly-5-hydroxyvalerate. The copolymer is a poly-3-hydroxybutyrate copolymer, for example a poly-3-hydroxybutyrate-co-4-hydroxybutyrate copolymer or a poly-3-hydroxybutyrate-co-5-hydroxyvalerate copolymer, in particular, a poly-3-hydroxybutyrate-co-4-hydroxybutyrate having 10% 4-hydroxybutyrate, poly-3-hydroxybutyrate-co-4-hydroxybutyrate having 50% 4-hydroxybutyrate, poly-3-hydroxybutyrate-co-4-hydroxybutyrate having 90% 4-hydroxybutyrate poly-3-hydroxybutyrate-co-5-hydroxyvalerate having 10% 5-hydroxyvalerate, poly-3-hydroxybutyrate-co-5-hydroxyvalerate having 50% 5-hydroxyvalerate, and poly-3-hydroxybutyrate-co-5-hydroxyvalerate having 90% 5-hydroxyvalerate.

In a further embodiment, monomer is further enzymatically processed to a diol for example, the monomer is 3-hydroxypropionate and the diol is 1,3-propanediol, or the monomer is 4-hydroxybutyrate and the diol is 1,4 butanediol, or the monomer is 5-hydroxyvalerate and the diol is 1,5 pentanediol.

In a further embodiment, monomer is further processed to a di-acid or lactone for example, the monomer is 5-hydroxyvalerate and the di-acid is glutarate and the lactone is delta-valerolactone.

In other embodiments, the pathway is an autotropic 3-hydroxypropionate carbon fixation pathway via a substrate selected from malonyl-CoA, glycerol and beta-alanine

In any of the embodiments or aspects of the invention, under aerobic conditions or under anaerobic conditions and optionally includes a hydrogen co-feed and/or optionally a carbon dioxide co-feed.

In certain aspects, the method includes reducing carbon dioxide in the pathway.

The organism for the methods or organism of the invention is selected from Escherichia coli, Ralstonia eutropha (Cupravidus necator, Alcaligenes eutrophus), Metallosphaera sedula, Sulfolobus genus, Pyrobaculum genus, Caldivirga maquilingensis, Thermoproteus neutrophilus, Acinetobacter baumannii, Acinetobacter baylyi, Acinetobacter aceti, Acinetobacter sp. DR1, Acinetobacter calcoaceticus, Acinetobacter haemolyticus, Acinetobacter johnsonii, Acinetobacter junii, Acinetobacter lwoffii, Acinetobacter radioresistens, Acinetobacter venetianus, Acinetobacter sp. DSM, Zoogloea ramigera, Allochromatium vinosum, Rhodococcus ruber, Delftia acidovorans, Aeromonas caviae, Synechocystis sp. PCC 6803, Synechococcus elongatus PCC 7942, Thiocapsa pfenigii, Bacillus megaterium, Clostridium kluyveri, Methylobacterium extorquens, Nocardia corralina, Nocardia salmonicolor, Pseudomonas fluorescens, Pseudomonas oleovorans, Pseudomonas sp. 6-19, Pseudomonas sp.61-3 and Pseudomonas putida, Rhodobacter sphaeroides, Alcaligenes latus, Klebsiella oxytoca, Anaerobiospirillum succiniciproducens, Actinobacillus succinogenes, Mannheimia succiniciproducens, Rhizobium etli, Bacillus subtilis, Corynebacterium glutamicum, Gluconobacter oxydans, Zymomonas mobilis, Lactococcus lactis, Lactobacillus plantarum, Streptomyces coelicolor, Clostridium acetobutylicum, Saccharomyces cerevisiae, Schizosaccharomyces pombe, Kluyveromyces lactis, Kluyveromyces marxianus, Aspergillus terreus, Aspergillus niger, Pichia pastoris, Chlorella spp., Chlorella minutissima, Chlorella emersonii, Chlorella sorokiniana, Chlorella ellipsoidea, Chlorella sp., and Chlorella protothecoides.

In the methods described herein wherein the pathway (either a PHA pathway and/or a carbon fixation pathway) incorporated includes at least one genetically modified gene encoding an enzyme. For example, in certain methods the organism has a 3-hydroxypropionate bi-cycle carbon fixation pathway that includes genetic modification of a gene of at least one of the following enzymes: (1) Acetyl-CoA carboxylase; (2) malonyl-CoA reductase; (3) propionyl-CoA synthase; (4) propionyl-CoA carboxylase; (5) methylmalonyl-CoA epimerase; (6) methylmalonyl-CoA mutase; (7) succinyl-CoA:(S)-malate-CoA transferase; (8) succinate dehydrogenase; (9) fumarate hydratase; (10 a, b, c) (S)-malyl-CoA/β-methylmalyl-CoA/(S)-citramalyl-CoA (MMC) lyase; (11) mesaconyl-C1-CoA hydratase (β-methylmalyl-CoA dehydratase); (12) mesaconyl-CoA C1-C4 CoA transferase; and (13) mesaconyl-C4-CoA hydratase, or the genes of at least two, at least three, at least four, at least five, at least six, at least seven, at least eight, at least nine, at least ten, at least eleven, at least twelve, at least thirteen, at least fourteen, at least fifteen, at least sixteen or all of the enzymes in the pathway are genetically modified.

In another embodiment, the carbon fixation pathway is 3-hydroxypropionate/4-hydroxybutyrate pathway and the gene of at least one of the following enzymes is genetically modified: (1) acetyl-CoA carboxylase; (2) malonyl-CoA reductase (NADPH); (3) malonate semialdehyde reductase (NADPH); (4) 3-hydroxypropionyl-CoA synthetase (AMP-forming); (5) 3-hydroxypropionyl-CoA dehydratase; (6) acryloyl-CoA reductase (NADPH); (7) propionyl-CoA carboxylase; (8) methylmalonyl-CoA epimerase; (9) methylmalonyl-CoA mutase; (10) succinyl-CoA reductase (NADPH); (11) succinate semialdehyde reductase (NADPH); (12) 4-hydroxybutyryl-CoA synthetase (AMP-forming); (13) 4-hydroxybutyryl-CoA dehydratase; (14) crotonyl-CoA hydratase; (15) 3-hydroxybutyryl-CoA dehydrogenase (NAD+); and (16) acetoacetyl-CoA β-ketothiolase.

Also contemplated by the invention are organisms homologously having a carbon fixation pathway capable of utilizing glucose (or other sugars such as sucrose, sugars derived from cellulosic hydrolysates and the like) as a carbon source, wherein the organism is genetically engineered to incorporate a polyhydroxyalkanoate pathway for producing a polyhydroxyalkanoate monomer, polymer or copolymer; organisms homologously having a carbon fixation pathway capable of utilizing glucose (or other sugars such as sucrose, sugars derived from cellulosic hydrolysates and the like) as a carbon source, wherein the organism is genetically engineered to incorporate a lysine pathway for producing lysine; organisms homologously capable of producing a polyhydroxyalkanoate polymer, wherein the organism is genetically engineered to incorporate a carbon fixation pathway to utilize a carbon feedstock for example, glucose (or other sugars such as sucrose, sugars derived from cellulosic hydrolysates and the like) and optionally incorporating a hydrogen gas co-feed and optionally carbon dioxide co-feed and wherein the organism produces an increased yield over an organism without the carbon fixation pathways; and organisms genetically engineered to incorporate a polyhydroxyalkanoate pathway and a carbon fixation pathway.

In a further embodiment, a process is described for producing a diol, comprising: providing an organism capable of producing diol; genetically engineering the organism incorporating a carbon fixation pathway to convert glucose (or other sugars such as sucrose, sugars derived from cellulosic hydrolysates and the like) to acetyl-CoA when grown on glucose (or other sugars such as sucrose, sugars derived from cellulosic hydrolysates and the like) as a carbon source, thereby producing a diol-producing organism genetically engineered to utilize glucose (or other sugars such as sucrose, sugars derived from cellulosic hydrolysates and the like); and providing glucose (or other sugars such as sucrose, sugars derived from cellulosic hydrolysates and the like) to the diol-producing organism genetically engineered to utilize glucose (or other sugars such as sucrose, sugars derived from cellulosic hydrolysates and the like); thereby producing diol and for producing a diol, comprising: providing an organism incorporating a carbon fixation pathway to convert glucose (or other sugars such as sucrose, sugars derived from cellulosic hydrolysates and the like) to acetyl-CoA when grown on glucose (or other sugars such as sucrose, sugars derived from cellulosic hydrolysates and the like) as a carbon source, incorporating a diol pathway thereby producing a diol-producing organism genetically engineered to utilize glucose (or other sugars such as sucrose, sugars derived from cellulosic hydrolysates and the like); and providing glucose (or other sugars such as sucrose, sugars derived from cellulosic hydrolysates and the like) to the diol-producing organism genetically engineered to utilize glucose (or other sugars such as sucrose, sugars derived from cellulosic hydrolysates and the like); thereby producing diol.

In certain embodiments, wherein the organism is E. coli, and the PHA pathway is poly-3-hydroxybutyrate (P3HB), wherein the P3HB is produced under anaerobic conditions or PHA pathway is a poly 3-hydroxybutyrate pathway and the method is under aerobic conditions optionally incorporating a hydrogen co-feed or a carbon dioxide co-feed.

In other embodiments, the organism is E. coli, the PHA pathway is poly-4-hydroxybutyrate and the method is under anaerobic conditions and optionally further incorporating a hydrogen co-feed and optionally a carbon dioxide co-feed.

In other embodiments, organism is E. coli, the PHA pathway is poly-5-hydroxyvalerate and the method is under aerobic conditions and includes a hydrogen co-feed and optionally a carbon-dioxide co-feed.

In yet another embodiment, the organism is E. coli, the PHA pathway is a poly-3-hydroxypropionoate pathway via a substrate selected from malonyl-coA, glycerol and beta-alanine, and the method is under aerobic conditions including a hydrogen co-feed and optionally a carbon dioxide co-feed or under anaerobic conditions including a hydrogen co-feed and a carbon dioxide co-feed.

In still other embodiment, the organism is E. coli, the PHA pathway is a poly-3-hydroxybutyrate-co-4-hydroxybutyrate polymer pathway and the method is under aerobic conditions including a hydrogen co-feed and optionally a carbon dioxide co-feed.

In yet other embodiments, the organism is E. coli, the PHA pathway results is a 1,4 butanediol product and the method is under aerobic conditions including a hydrogen co-feed and optionally a carbon dioxide co-feed or the PHA pathway results is a 1,3 propanediol product and the method is under aerobic conditions including a hydrogen co-feed and optionally a carbon dioxide co-feed.

The invention also pertains to an organism homologously having a carbon fixation pathway capable of utilizing glucose as a carbon source, wherein the organism is genetically engineered to incorporate a polyhydroxyalkanoate pathway for producing a polyhydroxyalkanoate monomer, polymer or copolymer, an organism homologously having a carbon fixation pathway capable of utilizing sucrose or a sugar derived from a cellulosic hydrolysate as a carbon source, wherein the organism is genetically engineered to incorporate a lysine pathway for producing lysine, an organism homologously capable of producing a polyhydroxyalkanoate polymer, wherein the organism is genetically engineered to incorporate a carbon fixation pathway to utilize sucrose or a sugar derived from a cellulosic hydrolysate and optionally incorporating a hydrogen gas pathway and optionally carbon dioxide pathway and wherein the organism produces an increased yield over an organism with out the carbon fixation pathways, an organism genetically engineered to incorporate a polyhydroxyalkanoate pathway and a carbon fixation pathway, an organism having a polyhydroxyalkanoate pathway and a carbon fixation pathway and genetically modified to utilize glucose, sucrose or a sugar derived from a cellulosic hydrosylate as a carbon source, wherein the organism produces an increased yield of PHA over an organism with out the genetic modification, and an organism having a polyhydroxyalkanoate pathway, a carbon fixation pathway and utilizes glucose, sucrose or a sugar derived from a cellulosic hydrosylate as a carbon source, wherein the organism has been genetically modified via promoter and regulatory system modification for producing an increased yield of PHA over an organism with out the genetic modification.

In another aspect of the invention, methods and organism are described further including genetically incorporating or up regulating a hydrogenase gene for hydrogen uptake.

### BRIEF DESCRIPTION OF THE DRAWINGS

FIG. 1 is a schematic of the P3HB pathway via acetyl-CoA.

FIG. 2 is a schematic of the P4HB pathway via succinyl-CoA.

FIG. 3 is a schematic of the P5HV pathway via lysine.

FIG. 4 is a schematic of the P(3HB-co-4HB) pathway via acetyl-CoA and succinyl-CoA.

FIG. 5 is a schematic of the P(3HB-co-5HV) pathway via acetyl-CoA and lysine.

FIG. 6 is a schematic of the P3HP pathway via malonyl-CoA (“P3HP-mcr”).

FIG. 7 is a schematic of the 3-hydroxypropionate/4-hydroxybutyrate CO₂ fixation pathway in Metallosphaera sedula. Proposed autotrophic 3-hydroxypropionate/4-hydroxybutyrate cycle in M. sedula. Reactions of the cycle are shown. Enzymes: 1) acetyl-CoA carboxylase; 2) malonyl-CoA reductase (NADPH); 3) malonate semialdehyde reductase (NADPH); 4) 3-hydroxypropionyl-CoA synthetase (AMP-forming); 5) 3-hydroxypropionyl-CoA dehydratase; 6) acryloyl-CoA reductase (NADPH); 7) propionyl-CoA carboxylase; 8) methylmalonyl-CoA epimerase; 9) methylmalonyl-CoA mutase; 10) succinyl-CoA reductase (NADPH); 11) succinate semialdehyde reductase (NADPH); 12) 4-hydroxybutyryl-CoA synthetase (AMP-forming); 13) 4-hydroxybutyryl-CoA dehydratase; 14) crotonyl-CoA hydratase; 15) 3-hydroxybutyryl-CoA dehydrogenase (NAD+); 16) acetoacetyl-CoA P ketothiolase.

FIG. 8 is a schematic of the 3-hydroxypropionate CO₂ fixation pathway in Chloroflexus aurantiacus. The complete 3-hydroxypropionate cycle, as studied in C. aurantiacus. [1] Acetyl-CoA carboxylase, [2] malonyl-CoA reductase, [3] propionyl-CoA synthase, [4] propionyl-CoA carboxylase, [5] methylmalonyl-CoA epimerase, [6] methylmalonyl-CoA mutase, [7] succinyl-CoA:(S)-malate-CoA transferase, [8] succinate dehydrogenase, [9] fumarate hydratase, [10 a, b, c] (S)-malyl-CoA/β-methylmalyl-CoA/(S)-citramalyl-CoA (MMC) lyase, [11] mesaconyl-C1-CoA hydratase (β-methylmalyl-CoA dehydratase), [12] mesaconyl-CoA C1-C4 CoA transferase, [13] mesaconyl-C4-CoA hydratase.

### DETAILED DESCRIPTION OF THE INVENTION

A description of example embodiments of the invention follows.

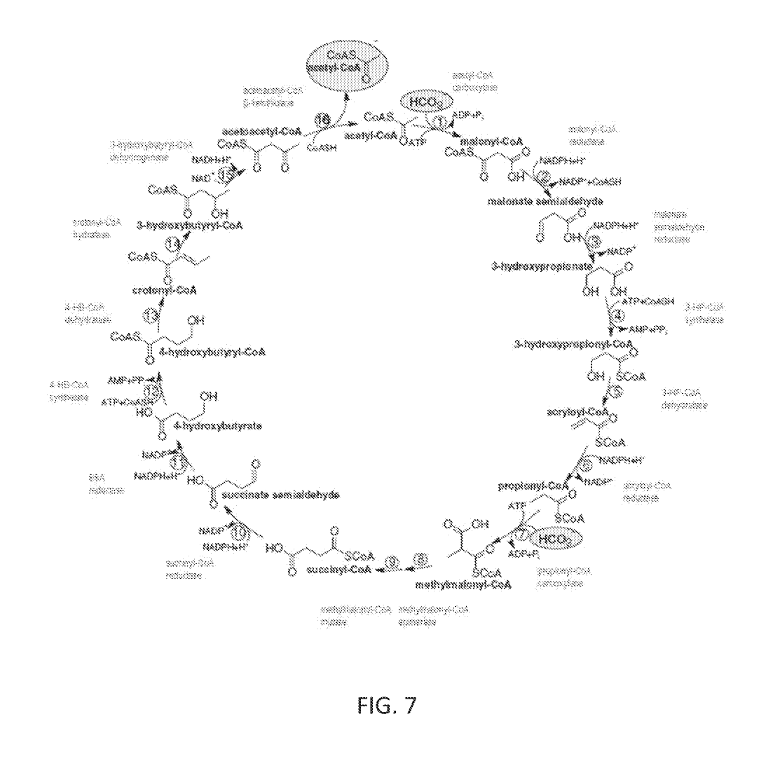

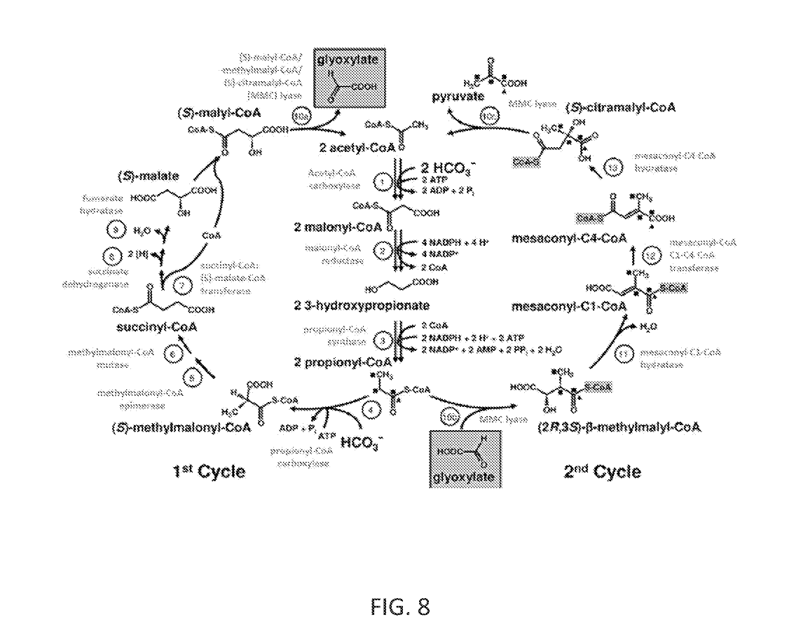

Described herein are methods of utilizing at least one enzyme of a carbon fixation pathway in combination with a PHA pathway for yield increases of PHA products. Two pathways of carbon fixation for the methods and organisms described herein include the 3-Hydroxyproprionate (3HP) bi-cycle (Zarzycki (2009) et. al., PNAS, vol. 106, No. 50, p 21317) found in Chloroflexus aurantiacus and the 3-Hydroxypropionate-4-Hydroxybutyrate (3HP/4HB) cycle found in Metallospharea sedula. See FIGS. 7 and 8.

The term “PHA copolymer” refers to a polymer composed of at least two different hydroxyalkanoic acid monomers.

The term “PHA homopolymer” refers to a polymer that is composed of a single hydroxyalkanoic acid monomer.

As used herein, a “vector” is a replicon, such as a plasmid, phage, or cosmid, into which another DNA segment may be inserted so as to bring about the replication of the inserted segment. The vectors can be expression vectors.

As used herein, an “expression vector” is a vector that includes one or more expression control sequences.

As used herein, an “expression control sequence” is a DNA sequence that controls and regulates the transcription and/or translation of another DNA sequence.

As used herein, “operably linked” means incorporated into a genetic construct so that expression control sequences effectively control expression of a coding sequence of interest.

As used herein, “transformed” and “transfected” encompass the introduction of a nucleic acid into a cell by a number of techniques known in the art.

As used herein “overproduced” means that the particular compound is produced at a higher quantity in the engineered organism as compared to the non-engineered organism.

As used herein the terms “renewable feedstock”, “renewable carbon substrate” and “renewable substrate” are all used interchangeably.

“Plasmids” are designated by a lower case “p” preceded and/or followed by capital letters and/or numbers.

As used herein, the term “heterologous” means that a gene or gene fragment encoding a protein is obtained from one or more sources other than the genome of the species within which it is ultimately expressed. The source can be natural, e.g., the gene can be obtained from another source of living matter, such as bacteria, yeast, fungi and the like, or a different species of plant. The source can also be synthetic, e.g., the gene or gene fragment can be prepared in vitro by chemical synthesis. “Heterologous” can also be used in situations where the source of the gene fragment is elsewhere in the genome of the plant in which it is ultimately expressed.

As used herein, to say that an organism is “homologously” capable of a biochemical reaction, means that the organism naturally possesses the genetic and cellular machinery to undertake the stated reaction. For instance, an organism that is homologously capable of converting glucose (or other sugars such as sucrose, sugars derived from cellulosic hydrolysates and the like) utilizing the carbon fixation pathway is an organism that naturally is capable of doing so. Similarly, an organism that is homologously capable of producing polyhydroxyalkanoate monomer or polymer is an organism that is naturally capable of producing such monomers and polymers.

A “diol” is a chemical compound containing two hydroxyl (—OH) groups.

A “di-acid” is a chemical compound containing two carboxylic acid (—COOH) groups.

A “higher alcohol” (or secondary alcohol) is an alcohol containing more than two carbons.

As used herein “yield” refers to the amount of product per amount of carbon source (g/g or wt/wt). The maximal theoretical yield calculated by various techniques provides the greatest (maximum) yield (wt/wt) for any given biochemical process from carbon source to end-product. See below for examples.

### Suitable Extrachromosomal Vectors and Plasmids

A “vector,” as used herein, is an extrachromosomal replicon, such as a plasmid, phage, or cosmid, into which another DNA segment may be inserted so as to bring about the replication of the inserted segment. Vectors vary in copy number and depending on the origin of their replication they contain, in their size, and the size of insert. Vectors with different origin of replications can be propagated in the same microbial cell unless they are closely related such as pMB1 and ColE1. Suitable vectors to express recombinant proteins can constitute pUC vectors with a pMB1 origin of replication having 500-700 copies per cell, pBluescript vectors with a ColE1 origin of replication having 300-500 copies per cell, pBR322 and derivatives with a pMB 1 origin of replication having 15-20 copies per cell, pACYC and derivatives with a p15A origin of replication having 10-12 copies per cell, and pSC101 and derivatives with a pSC101 origin of replication having 2-5 copies per cell as described in the QIAGEN® Plasmid Purification Handbook (found on the world wide web at: //kirshner.med.harvard.edu/files/protocols/QIAGEN_QIAGENPlasmidPurification_EN.pdf). Another useful vector is the broad host-range cloning vector pBBR1MCS with a pBBR1 origin of replication and its derivatives that contain different antibiotic resistance cassettes (Kovach et al., Gene 166:175-176 (1995)). These vectors are compatible with IncP, IncQ and IncW group plasmids, as well as with ColE1- and p15A-based replicons. A widely used vector is pSE380 that allows recombinant gene expression from an IPTG-inducible trc promoter (Invitrogen, La Jolla, Calif.).

### Suitable Strategies and Expression Control Sequences for Recombinant Gene Expression

Strategies for achieving expression of recombinant genes in E. coli have been extensively described in the literature (Gross, Chimica Oggi 7(3):21-29 (1989); Olins and Lee, Cur. Op. Biotech. 4:520-525 (1993); Makrides, Microbiol. Rev. 60(3):512-538 (1996); Hannig and Makrides, Trends in Biotech. 16:54-60 (1998)). Expression control sequences can include constitutive and inducible promoters, transcription enhancers, transcription terminators, and the like which are well known in the art. Suitable promoters include, but are not limited to, P_{lac}, P_{tac}, P_{trc}, P_{R}, F_{L}, Pₜᵣₚ, P_{phoA}, Pₐᵣₐ, P_{uspA}, P_{rspU}, Pₜₑₜ, P_{syn} (Rosenberg and Court, Ann. Rev. Genet. 13:319-353 (1979); Hawley and McClure, Nucleic Acids Res. 11 (8):2237-2255 (1983); Harley and Raynolds, Nucleic Acids Res. 15:2343-2361 (1987); also ecocyc.org and partsregistry.org). Exemplary promoters are:

P_{syn1} (a.k.a. P_{synA})
SEQ ID NO: 1
(5′-TTGACAGCTAGCTCAGTCCTAGGTATAATGCTAGC-3′),
P_{synC}
SEQ ID NO: 2
(5′-TTGACAGCTAGCTCAGTCCTAGGTACTGTGCTAGC-3′),
P_{synE}
SEQ ID NO: 3
(5′-TTTACAGCTAGCTCAGTCCTAGGTATTATGCTAGC-3′),
P_{synH}
SEQ ID NO: 4
(5′-CTGACAGCTAGCTCAGTCCTAGGTATAATGCTAGC-3′),
P_{synK}
SEQ ID NO: 5
(5′-TTTACGGCTAGCTCAGTCCTAGGTACAATGCTAGC-3′),
P_{synM}
SEQ ID NO: 6
(5′-TTGACAGCTAGCTCAGTCCTAGGGACTATGCTAGC-3′),
P_{trc}
SEQ ID NO: 7
(5′-TTGACAATTAATCATCCGGCTCGTATAATG-3′),
P_{tac}
SEQ ID NO: 8
(5′-TTGACAATTAATCATCGTCGTATAATGTGTGGA-3′),
Pₜₑₜ
SEQ ID NO: 9
(5′-TCCCTATCAGTGATAGAGATTGACATCCCTATCAGTGATAGAGATACTGAGCAC-3′),
Pₓ
SEQ ID NO: 10
(5′-TCGCCAGTCTGGCCTGAACATGATATAAAAT-3′),
P_{uspA}
SEQ ID NO: 11
(5′-AACCACTATCAATATATTCATGTCGAAAATTTGTTTATCTAACGAGTAAGCAAGGC
GGATTGACGGATCATCCGGGTCGCTATAAGGTAAGGATGGTCTTAACACTGAATC
CTTACGGCTGGGTTAGCCCCGCGCACGTAGTTCGCAGGACGCGGGTGACGTAACG
GCACAAGAAACG-3′),
P_{rpsU}
SEQ ID NO: 12
(5′-ATGCGGGTTGATGTAAAACTTTGTTCGCCCCTGGAGAAAGCCTCGTGTATACTCCT
CACCCTTATAAAAGTCCCTTTCAAAAAAGGCCGCGGTGCTTTACAAAGCAGCAGC
AATTGCAGTAAAATTCCGCACCATTTTGAAATAAGCTGGCGTTGATGCCAGCGGCA
AAC-3′).
P_{synAF7}
SEQ ID NO: 13
(5′-TTGACAGCTAGCTCAGTCCTAGGTACAGTGCTAGC-3′)
P_{synAF3}
SEQ ID NO: 14
(5′-TTGACAGCTAGCTCAGTCCTAGGTACAATGCTAGC-3′)

Exemplary terminators are:

T_{trpL}
SEQ ID NO: 15
(5-CTAATGAGCGGGCTTTTTTTTGAACAAAA-3′),
T₁₀₀₆
SEQ ID NO: 16
(5-AAAAAAAAAAAACCCCGCTTCGGCGGGGTTTTTTTTTT-3′),
T_{rrnB1}
SEQ ID NO: 17
(5-ATAAAACGAAAGGCTCAGTCGAAAGACTGGGCCTTTCGTTTTA
T-3′),
T_{rrnB2}
SEQ ID NO: 18
(5-AGAAGGCCATCCTGACGGATGGCCTTTT-3′).

### Construction of Recombinant Hosts

Recombinant hosts containing the necessary genes that will encode the enzymatic pathway for the conversion of a carbon substrate such as e.g. glucose (or other sugars such as sucrose, sugars derived from cellulosic hydrolysates and the like) to PHA and chemicals may be constructed using techniques well known in the art.

Methods of obtaining desired genes from a source organism (host) are common and well known in the art of molecular biology. Such methods can be found described in, for example, Sambrook et al., Molecular Cloning. A Laboratory Manual, Third Ed., Cold Spring Harbor Laboratory, New York (2001); Ausubel et al., Current Protocols in Molecular Biology, John Wiley and Sons, Baltimore, Md. (1999). For example, if the sequence of the gene is known, the DNA may be amplified from genomic DNA using polymerase chain reaction (Mullis, U.S. Pat. No. 4,683,202) with primers specific to the gene of interest to obtain amounts of DNA suitable for ligation into appropriate vectors. Alternatively, the gene of interest may be chemically synthesized de novo in order to take into consideration the codon bias of the host organism to enhance heterologous protein expression. Expression control sequences such as promoters and transcription terminators can be attached to a gene of interest via polymerase chain reaction using engineered primers containing such sequences. Another way is to introduce the isolated gene into a vector already containing the necessary control sequences in the proper order by restriction endonuclease digestion and ligation. One example of this latter approach is the BIOBRICK™ technology (see the world wide web at biobricks.org) where multiple pieces of DNA can be sequentially assembled together in a standardized way by using the same two restriction sites.

In addition to using vectors, genes that are necessary for the enzymatic conversion of a carbon substrate such as e.g. sucrose to PHA and chemicals can be introduced into a host organism by integration into the chromosome using either a targeted or random approach. For targeted integration into a specific site on the chromosome, the method generally known as Red/ET recombineering is used as originally described by Datsenko and Wanner (Proc. Natl. Acad. Sci. USA, 97:6640-6645 (2000)). Random integration into the chromosome involved using a mini-Tn5 transposon-mediated approach as described by Huisman et al. (U.S. Pat. Nos. 6,316,262 and 6,593,116).

Using the engineering methods described herein, one of ordinary skill in the art can (1) start with an organism comprising a carbon fixation pathway and engineer it to make useful products as described herein, (2) start with an organism capable of making useful products and engineer it to include a carbon fixation pathway, or (3) start with an organism, and engineer it to include both pathways as described herein.

For instance, one can start with an organism having a carbon fixation pathway, and engineer it according to methods described herein to make useful products. Alternatively, one can start with an organism such as Ralstonia eutropha, which is known to make polyhydroxyalkanoate polymers, and engineer it to include a carbon fixation pathway Also shown below are microbes that neither have a carbon fixation pathway or synthesize polyhydroxyalkanoates naturally, and are engineered to include both pathways and increase yields of the products.

Methods of culturing such engineered organisms to produce useful products are known in the art. To make some products, co-feeds besides glucose (or other sugars such as sucrose, sugars derived from cellulosic hydrolysates and the like) may be required, for instance, depending on the pathway(s) engineered into the organism, a co-feed of hydrogen or carbon dioxide or other substrate in the pathway are further included.

### Suitable Host Strains

Recombinant organisms having enzymes for the biochemical pathways to convert glucose (or other sugars such as sucrose, sugars derived from cellulosic hydrolysates and the like) to acetyl-CoA, and/or to produce useful products such as PHAs, diols, diacids, and higher alcohols, are provided. Host strains are genetically engineered to express the enzymes necessary to accomplish the metabolism of glucose (or other sugars such as sucrose, sugars derived from cellulosic hydrolysates and the like) as a substrate, and the production of such useful products.

The host strain can be a bacterium, a fungus, an alga, or other microbe. Organisms of cells that can be modified for production of PHAs, diols, diacids and higher alcohols include prokaryotes and eukaryotes. Suitable prokaryotes include bacteria.

The host strain can be, for example, Escherichia coli. In certain embodiments, the host strain is E. coli K-12 strain LS5218 (Sprat et al., J. Bacteriol. 146 (3):1166-1169 (1981); Jenkins and Nunn, J. Bacteriol. 169 (1):42-52 (1987)) or DH5α, (Raleigh et al., In: Ausubel et al., (Eds.) Current Protocols in Molecular Biology, p. 14 New York: Publishing Associates and Wiley Interscience (1989)). Other suitable E. coli K-12 host strains include, but are not limited to, MG1655 (Guyer et al., Cold Spr. Harb. Symp. Quant. Biol. 45:135-140 (1981)), WG1 and W3110 (Bachmann Bacteriol. Rev. 36(4):525-57 (1972)). Alternatively, E. coli strain W (Archer et al., BMC Genomics 2011, 12:9 doi:10.1186/1471-2164-12-9) or E. coli strain B (Delbruck and Luria, Arch. Biochem. 1:111-141 (1946)) and their derivatives such as REL606 (Lenski et al., Am. Nat. 138:1315-1341 (1991)) are other suitable E. coli host strains.

Other exemplary strains include Ralstonia eutropha (Cupravidus necator, Alcaligenes eutrophus, Metallosphaera sedula, Sulfolobus genus, Pyrobaculum genus, Caldivirga maquilingensis, Thermoproteus neutrophilus, Acinetobacter baumannii, Acinetobacter baylyi, Acinetobacter aceti, Acinetobacter sp. DR1, Acinetobacter calcoaceticus, Acinetobacter haemolyticus, Acinetobacter johnsonii, Acinetobacter junii, Acinetobacter twoffii, Acinetobacter radioresistens, Acinetobacter venetianus, Acinetobacter sp. DSM, Zoogloea ramigera, Allochromatium vinosum, Rhodococcus ruber, Delftia acidovorans, Aeromonas caviae, Synechocystis sp, PCC 6803, Synechococcus elongatus PCC 7942, Thiocapsa pfenigii, Bacillus megaterium, Clostridium kluyveri, Methylobacterium extorquens, Nocardia corralina, Nocardia salmonicolor, Pseudomonas fluorescens, Pseudomonas oleovorans, Pseudomonas sp. 6-19, Pseudomonas sp.61-3 and Pseudomonas putida, Rhodobacter sphaeroides, Alcaligenes latus, Klebsiella oxytoca, Anaerobiospirillum succiniciproducens, Actinobacillus succinogenes, Mannheimia succiniciproducens, Rhizobium etli, Bacillus subtilis, Corynebacterium glutamicum, Gluconobacter oxydans, Zymomonas mobilis, Lactococcus lactis, Lactobacillus plantarum, Streptomyces coelicolor, Clostridium acetobutylicum, Saccharomyces cerevisiae, Schizosaccharomyces pombe, Kluyveromyces lactis, Kluyveromyces marxianus, Aspergillus terreus, Aspergillus niger, Pichia pastoris, Chlorella spp., Chlorella minutissima, Chlorella emersonii, Chlorella sorokiniana, Chlorella ellipsoidea, Chlorella sp., and Chlorella protothecoides.

Exemplary yeasts or fungi include species selected from Saccharomyces cerevisiae, Schizosaccharomyces pombe, Kluyveromyces lactis, Kluyveromyces marxianus, Aspergillus terreus, Aspergillus niger and Pichia pastoris.

These include organisms that already produce polyhydroxyalkanoates, modified to utilize alternative substrates or incorporate additional monomers, or to increase production, and organisms that do not produce polyhydroxyalkanoates, but which express none to some of the enzymes required for production of polyhydroxyalkanoates. R. eutropha is an example of an organism which produces PHAs naturally. E. coli and C. glutamicum are examples of organisms where it would be necessary to introduce transgenes which encode the enzymes for PHA production.

Sources of encoding nucleic acids for utilizing a carbon fixation pathway include, for example, any species where the encoded gene product is capable of catalyzing one or more of the referenced reaction as described in FIGS. 7 and 8.

### Synthesis of Polyhydroxyalkanoate

During the mid-1980's, several research groups were actively identifying and isolating the genes and gene products responsible for PHA synthesis. These efforts led to the development of transgenic systems for production of PHAs in both microorganisms and plants, as well as enzymatic methods for PHA synthesis. Such routes could increase further the available PHA types. These advances have been reviewed in Williams & Peoples, CHEMTECH, 26:38-44 (1996) and Williams & Peoples, Chem. Br. 33:29-32 (1997).

Methods which can be used for producing PHA polymers suitable for subsequent modification to alter their rates of degradation are described, for example, in U.S. Pat. No. 4,910,145 to Holmes, et al.; Byrom, “Miscellaneous Biomaterials” in Biomaterials (Byrom, Ed.), pp. 333-59 (MacMillan Publishers, London 1991); Hocking & Marchessault, “Biopolyesters” in Chemistry and Technology of Biodegradable Polymers (Griffin, Ed.), pp. 48-96 (Chapman and Hall, London 1994); Holmes, “Biologically Produced (R)-3-hydroxyalkanoate Polymers and Copolymers,” in Developments in Crystalline Polymers (Bassett Ed.), vol. 2, pp. 1-65 (Elsevier, London 1988); Lafferty et al., “Microbial Production of Poly-b-hydroxybutyric acid” in Biotechnology (Rehm & Reed, Eds.) vol. 66, pp. 135-76 (Verlagsgesellschaft, Weinheim 1988); Muller & Seebach, Angew. Chem. Int. Ed. Engl. 32:477-502 (1993); Steinbuchel, “Polyhydroxyalkanoic Acids” in Biomaterials (Byrom, Ed.), pp. 123-213 (MacMillan Publishers, London 1991); Williams & Peoples, CHEMTECH, 26:38-44, (1996); Steinbuchel & Wiese, Appl. Microbiol. Biotechnol., 37:691-697 (1992); U.S. Pat. Nos. 5,245,023; 5,250,430; 5,480,794; 5,512,669; and 5,534,432; Agostini, et al., Polym. Sci., Part A-1, 9:2775-87 (1971); Gross, et al., Macromolecules, 21:2657-68 (1988); Dubois, et al., Macromolecules, 26:4407-12 (1993); Le Borgne & Spassky, Polymer, 30:2312-19 (1989); Tanahashi & Doi, Macromolecules, 24:5732-33 (1991); Hori, et al., Macromolecules, 26:4388-90 (1993); Kemnitzer, et al., Macromolecules, 26:1221-29 (1993); Hori, et al., Macromolecules, 26:5533-34 (1993); Hocking, et al., Polym. Bull., 30:163-70 (1993); Xie, et al., Macromolecules, 30:6997-98 (1997); U.S. Pat. No. 5,563,239 to Hubbs; U.S. Pat. Nos. 5,489,470 and 5,520,116 to Noda, et al. The PHAs derived from these methods may be in any form, including a latex or solid form.

Identification, cloning and expression of the genes involved in the biosynthesis of PHAs from several microorganisms within recombinant organisms allow for the production of PHAs within organisms that are not native PHA producers. A preferred example is E. coli, which is a well-recognized host for production of biopharmaceuticals, and PHAs for medical and other applications. Such recombinant organisms provide researchers with a greater degree of control of the PHA production process because they are free of background enzyme activities for the biosynthesis of unwanted PHA precursors or degradation of the PHA. Additionally, the proper selection of a recombinant organism may facilitate purification of, or allow for increased biocompatibility of, the produced PHA.

Polyhydroxyalkanoates (PHAs) are a form of biological carbon and energy storage compounds. These compounds are characterized by polyester repeat units of various chemical compositions. Many of these PHAs have shown to be useful as renewable feedstock for plastics goods and also a potentially valuable precursor for industrial chemicals.

Very often, organic carbon sources (sugars, alcohols, fatty acids, mixed sludge, etc.) are used as feeds for cultivation of PHA containing microorganisms. The yields of these processes are often limited by metabolic pathways for recycling carbon lost in the form of carbon dioxide and inefficient balancing of electrons. This document outlines methods which are calculated to relieve these constraints to increase yields for a variety of PHAs and small molecules from organic carbon substrates.

### Modeling of Metabolic Networks

Theoretical yields were calculated using a variety of techniques, including Elementary Mode Analysis, Flux Balance Optimizations, and Extreme Pathway analysis. These techniques have been well documented in the literature [Kauffman et al., Curr Opin Biotechnol 14(5):491-496 (2003); Schilling et al., Biotechnol Bioeng 71(4):286-306 (2000/2001); Trinh et al., Appl Microbiol Biotechnol 81:813-826 (2009)]. A model network based on the E. coli metabolic network was used as the foundation of subsequent changes associated with heterologous expression of substrate and product pathways, including PHA synthesis pathways, carbon recycle pathways, and hydrogen uptake routes. Genome scale metabolic networks of E. coli and other organisms are widely reported [Orth et al., Mol Sys Biol 7: Article 535 (2011)].

### Product Pathways

Several products were considered within the calculations presented here. A broad range of short-chain-length class polyhydroxyalkanoates are presented, including poly-3-hydroxybutyrate (P3HB), poly-4-hydroxybutyrate (P4HB), poly-5-hydroxyvalerate (P5HV), poly-3-hydroxypropionate (P3HP), as well as random copolymers of P3HB and P4HB (P3HB-x %-4HB, where x represents the percentage of 4HB in the random copolymer), and random copolymers of P3HB and P5HV (P3HB-x %-SHV, where x represents the percentage of 5HV in the random copolymer).

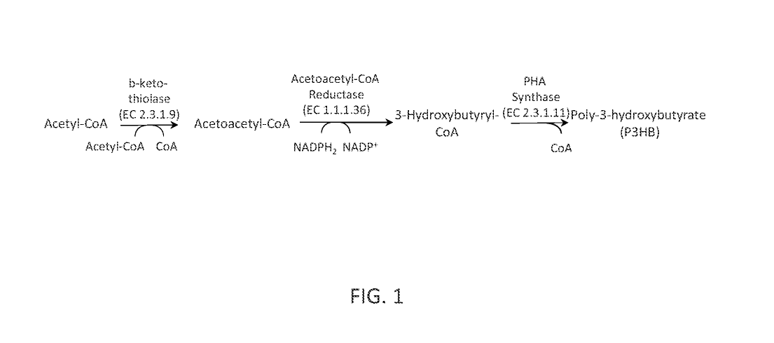

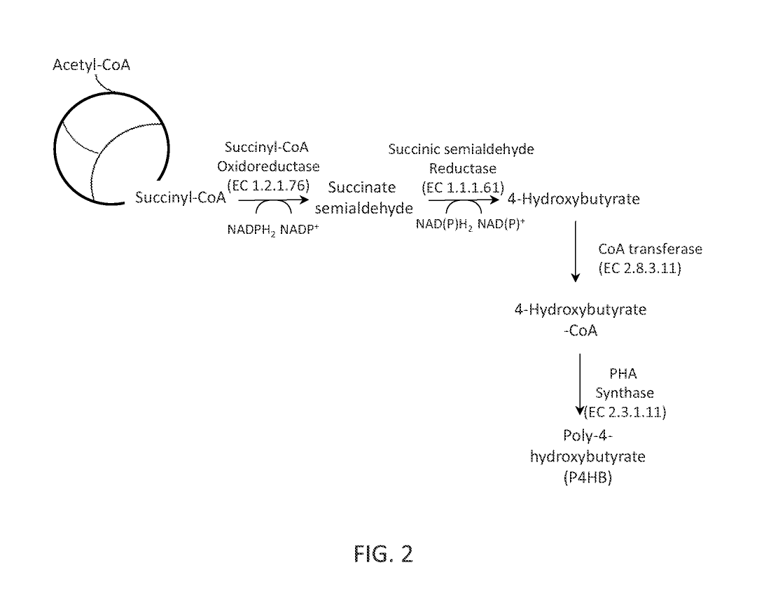

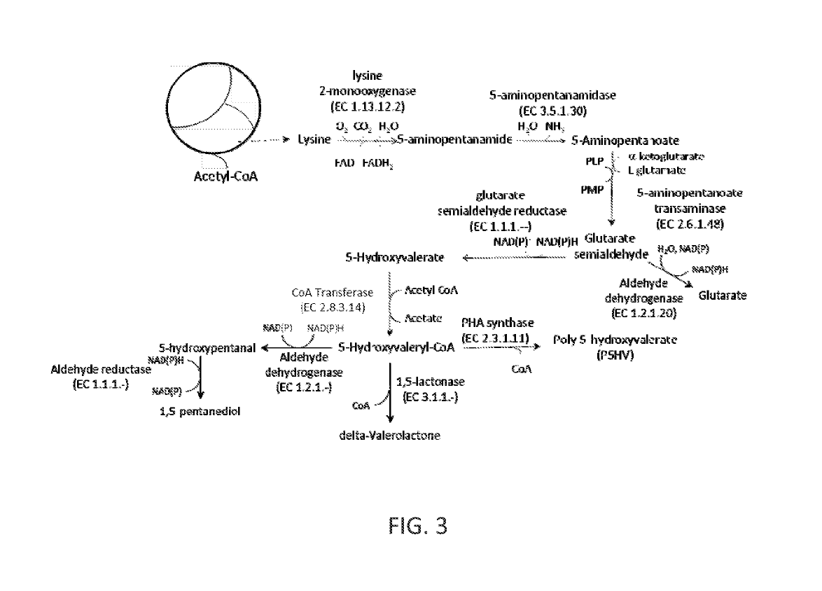

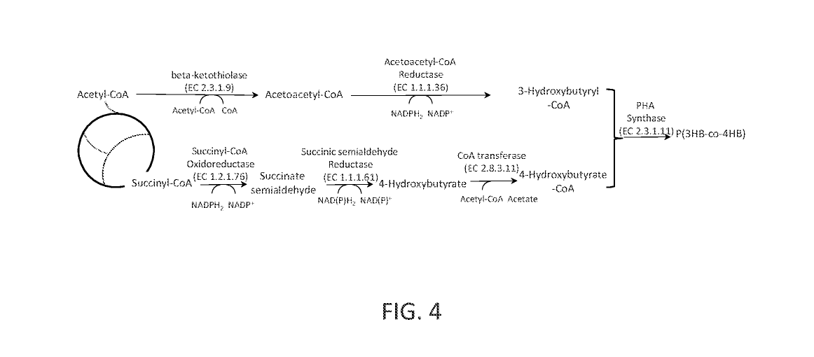

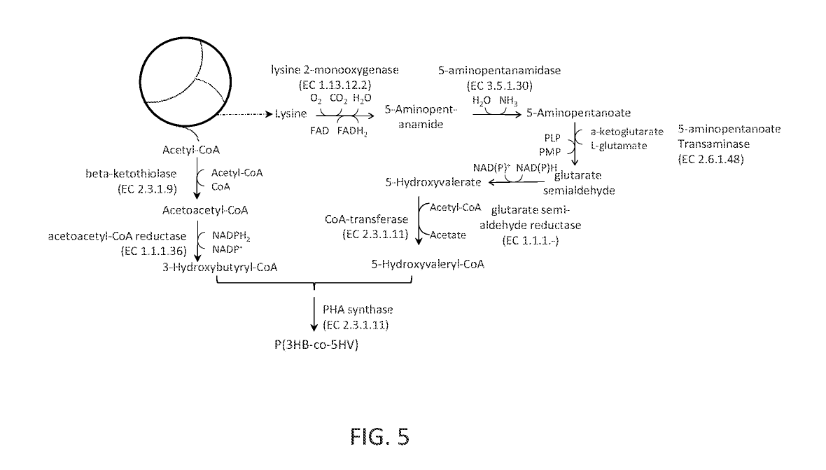

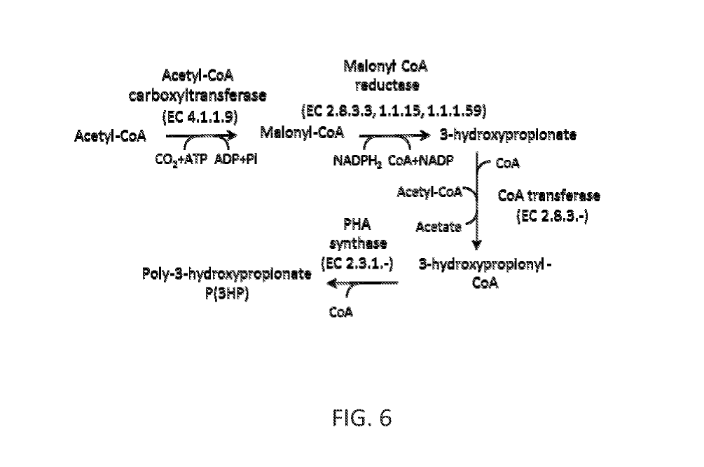

P3HB was synthesized via the pathway from acetyl-CoA presented previously [Peoples and Sinksey, J Biol Chem 264:15298-15303; Peoples and Sinskey, J Biol Chem 264:15293-15297] and here in FIG. 1. P4HB was synthesized via the pathway from succinyl-CoA presented previously [Van Walsem et al., WO Patent No. 2011/100601] and here in FIG. 2. P5HV was synthesized using the pathway from lysine presented previously Farmer et al., WO Patent No. 2010/068953] and here in FIG. 3. Copolymers of P3HB-4HB and P3HB-5HV are also investigated and presented in FIG. 4 and FIG. 5, respectively. P3HP was synthesized using three different routes, one via glycerol (“P3HP-gol” herein) presented previously [Skraly and Peoples, U.S. Pat. Nos. 6,329,183 and 6,323,010], another route via β-alanine (“P3HP-bAla” herein) presented previously [Gokarn et al., WO Patent No. 2002/042418A2], and a third route via malonyl-CoA (“P3HP-mcr” herein) presented previously [Gokarn et al., WO Patent No. 2002/042418] and here in FIG. 6.

The theoretical production of a variety of small molecules was also calculated. 1,3-propanediol was modeled using the glycerol route described previously [Laffend et al., WO Patent No. 1996/035796A1]. Further, use of 3-hydroxypropionate was modeled using the same routes as P3HP polymer, including the glycerol route (“3HP-gol” herein), β-alanine route (“3HP-bAla” herein), and malonyl-CoA route (“3HP-mcr” herein). 1,4-Butanediol was modeled using a previously described route [Van Dien et al., WO Patent No. 2010/141920A2] from succinyl-CoA. The production of 1,5-pentanediol, glutarate, and delta-valerolactone were modeled using derivatizations of the P5HV pathway as indicated here in FIG. 3. The overproduction of lysine was modeled using the endogenous E. coli route for lysine biosynthesis, which is already available in the genome scale model.

### Carbon Fixation Pathways

Two carbon fixation pathways were considered as alternatives for metabolic network augmentation. The first pathway is the 3HP/4HB autotrophic CO₂ fixation cycle. This cycle has been described previously [Berg et al., Science 318: 1782-1786 (2007)] in detail for the archaeal species Metallosphaera sedula. Other organisms, including many of the genus Sulfolobus, genus Pyrobaculum, Caldivirga maquilingensis, and Thermoproteus neutrophilus may contain this pathway, or components thereof. A diagram of the pathway is presented in FIG. 7. Table 1 contains enzyme commission numbers and overall stoichiometry reactions for each enzymatic reaction. Also locations in the M. sedula genome are reported which have been described as coding for enzymes with the specific desired functions. Within this document, the 3HP/4HB autotrophic carbon dioxide fixation cycle will be referred to as the “3HP/4HB cycle”.

TABLE 1
Details of enzymatic reactions for the completion of the 3HP/4HB autotrophic CO₂
fixation cycle. Each step is detailed with EC number, reaction description, reaction
stoichiometry, and example gene source for the desired enzyme.

| | EC | | | |
|---|---|---|---|---|
| Step | Number | Description | Reaction | Examples¹ |
| 1 | 6.4.1.2 | Acetyl-CoA carboxylase | acetyl-CoA + carbonate + ATP --> malonyl-CoA + | E. coli accB, M. sedula |
| | | | ADP + phosphate | Msed_0147/0148/1375 |
| 2 | 1.2.1.75 | Malonyl-CoA reductase | malonyl-CoA + NADPH --> malonyl-semialdehyde + | M. sedula Msed_0709 |
| | | | CoA + NADP+ | |
| 3 | 1.1.1.298 | Malonyl-semialdehyde reductase | malonyl-semialdehyde + NADPH | M. sedula Msed_1993 |
| | | | --> 3-hydroxypropionate + NADP+ | |
| 4 | 6.2.136 | 3-Hydroxypropionyl-CoA synthetase | 3-hydroxypropionate + CoA + ATP | M. sedula Msed_1456 |
| | | | --> 3-hydroxypropionyl-CoA + AMP + | |
| | | | diphosphate | |
| 5 | 4.2.1.116 | 3-Hydroxypropionyl-CoA | 3-hydroxypropionyl-CoA --> acrylyl-CoA + water | M. sedula Msed_2001 |
| | | dehydratase | | |
| 6 | 1.3.1.84 | Acrylyl-CoA Reductase | acrylyl-CoA + NADPH --> propanyl-CoA | M. sedula Msed_1426 |
| | 6.4.1.3 | Propanyl-CoA carboxylase | propanyl-CoA + carbonate + ATP | M. sedula Msed0147/0148/1375 |
| | | | --> (S)-methylmalonyl-CoA + ADP + phosphate | |
| 8 | 5.1.99.1 | Methylmalonyl-CoA epimerase | (S)-methylmalonyl-CoA --> (R)-methylmalonyl-CoA | M. sedula Msed_0639 |
| 9 | 5.4.99.2 | Methylmalonyl-CoA mutase | (R)-methylmalonyl-CoA --> succinyl-CoA | M. sedula Msed_0638/2055 |
| 10 | 1.2.1.76 | Succinyl-CoA reductase | succinyl-CoA + NADPH --> succinyl-semialdehyde + | M. sedula Msed_0709, 1774 |
| | | | NADP+ | |
| 11 | 1.11.— | Succinyl-semialdehyde reductase | succinyl-semialdehyde + NADPH --> 4-hydroxybutyrate + | M. sedula Msed_1424 |
| | | | NADP+ | |
| 12 | 6.2.1.— | 4-hydroxybutyryl-CoA synthetase | 4-hydroxybutyrate + CoA + ATP | M. sedula Msed_1422 |
| | | | --> 4-hydroxybutyryl-CoA + AMP + diphosphate | |
| 13 | 4.2.1.120 | 4-Hydroxybutyryl-CoA | 4-hydroxybutyryl-CoA --> vinyl acetyl-CoA + water | M. sedula Msed_1220/1321 |
| | | dehydratase | | |
| 14 | 5.3.3.3 | Vinylacetyl-CoA isomerase | vinylacetyl-CoA --> crotonyl-CoA | M. sedula Msed_1220/1321 |
| 15 | 4.2.1.55 | 3-Hydroxybutyryl-CoA | crotonyl-CoA + water --> 3-hydroxybutyryl-CoA | M. sedula Msed_0399 |
| | | dehydratase | | |
| 16 | 1.1.1.35 | 3-Hydroxybutyryl-CoA | 3-hydroxybutyryl-CoA + NAD + --> acetoacetyl- | M. sedula Msed_0399 |
| | | dehydrogenase | | CoA + NADH |
| 17 | 2.3.1.9 | Acetyl-CoA acetyltransferase | acetoacetyl-CoA + CoA --> 2 acetyl-CoA | M. sedula Msed_0270, |
| | | | | 0271, 0386, 0396, 0656 |

¹Comma separators “,” indicate alternative enzymes. Slash separators “/” indicate components of a heteromeric enzyme complex.

A second pathway is also examined for yield impacts. The autotrophic 3HP CO₂ fixation bi-cycle from Chloroflexus aurantiacus has been described previously [Zarzyck et al., PNAS 106 (50): 21317-21322 (2009)]. The steps of this pathway are described in Table 2 and FIG. 8. This table contains enzyme commission numbers describing each individual step as well as the overall prevailing stoichiometry reaction which is catalyzed by the enzymatic step. Table 2 also contains examples within the C. aurantiacus genome which may provide suitable genetic materials based on literature sources. Within this document, the autotrophic 3HP carbon dioxide fixation bi-cycle will be known as the “3HP Bi-Cycle”.

TABLE 2
Details of enzymatic reactions for the completion of the 3HP CO₂ fixation bi-cycle.
Each step is detailed with EC number, reaction description, reaction stoichiometry, and
example gene source for the desired enzyme.

| | EC | | | |
|---|---|---|---|---|
| Step | Number | Description | Reaction | Examples¹ |
| 1 | 6.4.1.2 | Acetyl-CoA carboxylase | acetyl-CoA + carbonate + ATP --> malonyl-CoA + | E. coli accB, C. aurantiacus |
| | | | ADP + phosphate | Caur_3799 |
| 2 | 1.2.1.75 | Malonyl-CoA reductase | malonyl-CoA + NADPH --> malonyl-semialdehyde + | C. aurantiacus Caur_2614 |
| | | | CoA + NADP+ | |
| 3 | 1.1.1.298 | Malonyl-semialdehyde reductase | malonyl-semialdehyde + NADPH | C. aurantiacus Caur_2614 |
| | | | --> 3-hydroxypropionate + NADP+ | |
| 4 | 6.2.1.36 | 3-Hydroxypropionyl-CoA synthetase | 3-hydroxypropionate + CoA + ATP | C. aurantiacus Caur_0613 |
| | | | --> 3-hydroxypropionyl-CoA + AMP + | |
| | | | diphosphate | |
| 5 | 4.2.1.116 | 3-Hydroxypropionyl-CoA dehydratase | 3-hydroxypropionyl-CoA --> acrylyl-CoA + water | C. aurantiacus Caur_0613 |
| 6 | 1.3.1.84 | Acrylyl-CoA Reductase | acrylyl-CoA + NADPH --> propanyl-CoA | C. aurantiacus Caur_0613 |
| 7 | 6.4.1.3 | Propanyl-CoA carboxylase | propanyl-CoA + carbonate + ATP | |
| | | | --> (S)-methylmalonyl-CoA + ADP + phosphate | C. aurantiacus Caur_2034 |
| 8 | 5.1.99.1 | Methylmalonyl-CoA epimerase | (S)-methylmalonyl-CoA --> (R)-methylmalonyl-CoA | C. aurantiacus Caur_3037 |
| 9 | 5.4.99.2 | Methylmalonyl-CoA mutase | (R)-methylmalonyl-CoA --> succinyl-CoA | C. aurantiacus Caur_2508, |
| | | | | Caur_2509 |
| 10 | SmtAB | Succinate:Malate CoA transferase | succinyl-CoA + malate --> succinate + malyl-CoA | C. aurantiacus Caur_0179 |
| 11 | 1.3.99.1 | Succinate dehydrogenase | succinate + FAD --> fumarate + FADH | E. coli sdhCDAB |
| 12 | 4.2.1.2 | Fumarate hydratase | fumarate + water --> malate | E. coli fumA, C. aurantiacus |
| | | | | Caur_1443 |
| 13 | SmtAB | Succinate:Malate CoA transferase | succinyl-CoA + malate --> succinate + malyl-CoA | C. aurantiacus Caur_0179 |
| 14 | 4.1.3.24 | Malyl-CoA lyase | malyl-CoA --> acetyl-CoA + glyoxylate | C. aurantiacus Caur_0174 (mcl) |
| 15 | 4.1.3.24 | Malyl-CoA lyase | propanyl-CoA + glyoxylate --> β-methylmalyl-CoA | C. aurantiacus Caur_0174 (mcl) |
| 16 | MCH | Mesaconyl-CoA hydratase | β-methylmalyl-CoA --> mesaconyl-C1-CoA + water | C. aurantiacus Caur_0173 (mch) |
| 17 | MCT | Mesaconyl-CoA mutase | mesaconyl-C1-CoA --> mesaconyl-C4-CoA | C. aurantiacus Caur_0180 (mct) |
| 18 | MEH | Mesaconyl-C4-CoA hydratase | mesaconyl-C4-CoA + water --> (3S)-citramalyl-CoA | C. aurantiacus Caur_0180 (meh) |
| 19 | 4.1.3.24 | Malyl-CoA lyase | (3S)-citramalyl-CoA --> acetyl-CoA + pyruyate | C. aurantiacus Caur_0174 (mcl) |

Other CO₂ fixation pathways to be considered to increase product yields include the Calvin-Benson Reductive Pentose Phosphate Cycle (Calvin, Nature 192(4805):799 (1961)), the Reductive Citric Acid (a.k.a. Arnon-Buchanan) Cycle (Evans et al., Proc. Natl. Acad. Sci. USA 55(4):928-934 (1966); Buchanan and Arnon, Photosynth. Res. 24:47-53 1990)), the Reductive Acetyl-CoA (a.k.a. the Wood-Ljungdahl) Pathway, and the Dicarboxylate/4-Hydroxybutyrate Cycle (Huber et al., Proc. Natl. Acad. Sci. USA 105:7851-7856 (2008). All six known CO₂ fixation pathways have been reviewed recently by Berg (Appl. Environ. Microbiol. 77(6): 1925-1936 (2011)).

Incorporation of the metabolic pathways into the overall network is described for both the utilization of only captive carbon dioxide (that which is created from heterotrophic cultivation) and also carbon dioxide which is supplied externally in excess for both pathways.

The chemical intermediates 3-hydroxypropionate, 3-hydroxypropionyl-CoA, 3-hydroxybutyrate-CoA and 4-hydroxybutyrate-CoA are important precursors for several of the product pathways presented above. When these intermediates are available in both the carbon fixation pathway, the simulations allow for the direct contribution of these intermediates from the carbon fixation pathway to the desired product synthesis in any ratio to the presented production formation pathway to optimize yield from glucose. Further, in the cases of the production of 1,3-propanediol, a reaction was included in the metabolic network which linked the 3HP intermediates of the carbon fixation cycles to the 3-hydroxyprionaldehyde (3HPA) in the PDO pathway under the stoichiometry reaction: 3HP+NADH→3HPA+NAD+. An example of an enzyme which may catalyze this reaction is E. coli AldH.

Other carbon fixation pathways may also be of interest. A method for identifying carbon fixation pathways, including novel synthetic carbon fixation pathways, has been previously described [Bar-Even et al., PNAS 107 (19):8889-8894 (2010)].

### Hydrogen Uptake Systems

Four hydrogenase isoenzymes have been identified in the E. coli genome (Self et al., J. Bacteriol. 186:580-587 (2004)). Two hydrogenases (hydrogenases 1 and 2) are involved in periplasmic hydrogen uptake, while the others (hydrogenases 3 and 4) are part of cytoplasmic formate hydrogenase complexes that evolve hydrogen (Sawers, Antonie van Leeuwenhoek 66:57-88 (1994); Self et al., J. Bacteriol. 186:580-587 (2004), Sawers, Biochem. Soc. Transac. 33:42-46 (2005); Vignais et al., FEMS Microbial. Rev. 25:455-501 (2001)). E. coli does not encode a soluble hydrogenase that couples oxidation of H₂ to the reduction of NAD⁺. Therefore, such an enzymatic reaction was added to the E. coli metabolic model. The hoxFUYHWI genes from Ralstonia eutropha for example encode such a soluble [NiFe]-hydrogenase (Burgdorf et al., J. Bacteriol. 184(22):6280-6288 (2002); Schwartz et al., J. Mol. Biol. 332:369-383 (2003); Burgdorf et al., J. Bacteriol. 187(9):3122-3132 (2005)).

### Basis for H₂ Feeding

Hydrogen gas can act as an efficient electron donor in cellular metabolism. Often, this feed is considered in the context of synthesis gas [Do et al., Biotechnol Bioeng 97 (2): 279-286 (2006)] (or syngas) or Knall-gas [Tanaka and Ishizaki, Biotechnol Bioeng 45: 268-275 (1995)] (a mixture of syngas with oxygen) fermentations. In these fermentations, electron-poor carbon sources (CO and/or CO₂) are combined with H₂ as an electron donor with sometimes oxygen as an additional electron acceptor to balance energy metabolisms. The result is chemolithoautotrophic metabolism.

In comparison, heterotrophic cultures rely on organic carbon sources (such as glucose) to supply both carbon and electrons to the metabolism. Oxygen can be used (and is sometimes necessary) to properly balance the metabolic pathway stoichiometry (by accepting excess hydrogen/electrons/etc.) according to pathway architecture constraints and energy requirements. Also, aerobic cultivation may help limit the production of unwanted toxic fermentation byproducts (acids, alcohols, etc.). Emissions of CO₂ are usually necessary and result in unwanted reductions in yields via carbon losses.

The theoretical yield analysis conducted herein utilizes hydrogen as a novel electron-source co-feed which is suitable to curb carbon losses and may facilitate carbon fixation for PHA products during organic substrate consumption. A model system for hydrogen utilization may be that of Ralstonia eutropha (a.k.a. Cupriavidus necator, Alcaligenes eutrophus) [Pohlmann et al., Nature Biotech 24 (10): 1257-1262], although many H₂ utilizing organisms are widely known. Typically a pathway for hydrogen assimilation may include a hydrogen dehydrogenase such as the hox complex from Ralstonia eutropha H16. A typical stoichiometric reaction for uptake may be H₂+NAD⁺→NADH+H⁺.

The examples presented herein provide theoretical yield calculations which demonstrate that hydrogen co-feeds or the archaeal carbon fixation pathway provide significant non-obvious and non-trivial improvements to the yields of PHAs from glucose. For some PHAs, a combination of both pathways sometimes provides further improvement over the individual modifications.

### EXAMPLES

TABLE 3
Summary of theoretical yield calculation results Calculations are presented for each
combination of product, substrate, aeration level, and presence of carbon fixation pathways.

| | Feeds | | | | | | | | |
|---|---|---|---|---|---|---|---|---|---|
| | Glucose | | | Glucose + H₂ | | | Glucose + H₂ + CO₂ | | |
| | Metabolic network | | | | | | | | |
| | Base | +3HB/4HB | +3HP | Base | +3HB/4HB | +3HP | Base | +3HB/4HB | +3HP |
| | Model | Cycle | Bi-Cycle | Model | Cycle | Bi-Cycle | Model | Cycle | Bi-Cycle |
| | Aeration | | | | | | | | |
| | Aerobic | | | | | | | | |
| P3HB | 0.478 | 0.631 | 0.589 | 0.478 | 0.717 | 0.717 | 0.478 | 0.956 | 0.956 |
| P4HB | 0.597 | 0.597 | 0.597 | 0.717 | 0.717 | 0.717 | 0.956 | 0.956 | 0.956 |
| P5HV | 0.437 | 0.437 | 0.439 | 0.556 | 0.667 | 0.667 | 0.556 | 1.111 | 1.111 |
| P3HP via mcr route | 0.649 | 0.649 | 0.615 | 0.800 | 0.800 | 1.200 | 0.800 | 1.600 | 2.000 |
| P3HP via Glycerol | 0.667 | 0.686 | 0.649 | 0.800 | 0.800 | 0.800 | 0.800 | 1.600 | 1.600 |
| Dehydratase | | | | | | | | | |
| P3HP via β-Alanine | 0.615 | 0.649 | 0.648 | 0.800 | 0.800 | 0.800 | 0.800 | 1.600 | 1.600 |
| P3HB-10%-4HB Copolymer | 0.503 | 0.630 | 0.597 | 0.503 | 0.717 | 0.717 | 0.503 | 0.956 | 0.956 |
| P3HB-50%-4HB Copolymer | 0.630 | 0.630 | 0.630 | 0.637 | 0.717 | 0.717 | 0.637 | 0.956 | 0.956 |
| P3HB-90%-4HB Copolymer | 0.601 | 0.601 | 0.601 | 0.717 | 0.717 | 0.717 | 0.869 | 0.956 | 0.956 |
| P3HB-10%-5HV Copolymer | 0.486 | 0.604 | 0.573 | 0.486 | 0.711 | 0.711 | 0.486 | 0.711 | 0.711 |
| P3HB-50%-5HV Copolymer | 0.517 | 0.520 | 0.520 | 0.517 | 0.689 | 0.689 | 0.517 | 0.689 | 0.689 |
| P3HB-90%-5HV Copolymer | 0.451 | 0.451 | 0.452 | 0.548 | 0.671 | 0.671 | 0.548 | 0.671 | 0.671 |
| 1,3-Propanediol | 0.618 | 0.633 | 0.633 | 0.844 | 0.844 | 0.844 | 0.844 | 1.689 | 1.689 |
| 3HP via mcr | 0.968 | 0.968 | 0.968 | 1.000 | 1.000 | 1.000 | 1.000 | 2.000 | 2.000 |
| 3HP via GolDH | 0.909 | 0.938 | 0.938 | 1.000 | 1.000 | 1.000 | 1.000 | 2.000 | 2.000 |
| 3HP via β-Alanine | 0.909 | 0.968 | 0.968 | 1.000 | 1.000 | 1.000 | 1.000 | 2.000 | 2.000 |
| 1,4-Butanediol | 0.546 | 0.546 | 0.546 | 0.750 | 0.750 | 0.750 | 1.000 | 1.000 | 2.000 |
| Lysine | 0.640 | 0.649 | 0.649 | 0.811 | 0.811 | 0.811 | 0.811 | 1.622 | 1.622 |
| | Aeration | | | | | | | | |
| | Anaerobic | | | | | | | | |
| P3HB | 0.000 | 0.618 | 0.494 | 0.000 | 0.618 | 0.494 | 0.000 | 0.618 | 0.542 |
| P4HB | 0.469 | 0.469 | 0.478 | 0.478 | 0.478 | 0.478 | 0.478 | 0.478 | 0.478 |
| P5HV | 0.000 | 0.000 | 0.000 | 0.000 | 0.000 | 0.000 | 0.000 | 0.000 | 0.000 |
| P3HP via mcr route | 0.313 | 0.313 | 0.313 | 0.313 | 0.313 | 0.267 | 0.360 | 0.360 | 0.733 |
| P3HP via Glycerol | 0.343 | 0.379 | 0.343 | 0.343 | 0.379 | 0.313 | 0.343 | 0.436 | 0.436 |
| Dehydratase | | | | | | | | | |
| P3HP via β-Alanine | 0.267 | 0.313 | 0.267 | 0.267 | 0.313 | 0.267 | 0.267 | 0.360 | 0.333 |
| P3HB-10%-4HB Copolymer | 0.368 | 0.604 | 0.531 | 0.368 | 0.604 | 0.531 | 0.382 | 0.604 | 0.557 |
| P3HB-50%-4HB Copolymer | 0.604 | 0.604 | 0.604 | 0.604 | 0.604 | 0.604 | 0.604 | 0.604 | 0.604 |
| P3HB-90%-4HB Copolymer | 0.499 | 0.499 | 0.499 | 0.499 | 0.499 | 0.499 | 0.499 | 0.499 | 0.499 |
| P3HB-10%-5HV Copolymer | 0.000 | 0.000 | 0.000 | 0.000 | 0.000 | 0.000 | 0.000 | 0.000 | 0.000 |
| P3HB-50%-5HV Copolymer | 0.000 | 0.000 | 0.000 | 0.000 | 0.000 | 0.000 | 0.000 | 0.000 | 0.000 |
| P3HB-90%-5HV Copolymer | 0.000 | 0.000 | 0.000 | 0.000 | 0.000 | 0.000 | 0.000 | 0.000 | 0.000 |
| 1,3-Propanediol | 0.563 | 0.633 | 0.633 | 0.563 | 0.633 | 0.633 | 0.563 | 0.633 | 0.633 |
| 3HP via mcr | 0.818 | 0.818 | 0.818 | 0.818 | 0.818 | 0.818 | 0.857 | 0.857 | 0.857 |
| 3HP via GolDH | 0.600 | 0.692 | 0.692 | 0.600 | 0.692 | 0.692 | 0.600 | 0.750 | 0.750 |
| 3HP via β-Alanine | 0.600 | 0.818 | 0.818 | 0.600 | 0.818 | 0.818 | 0.600 | 0.857 | 0.857 |
| 1,4-Butanediol | 0.546 | 0.546 | 0.546 | 0.546 | 0.546 | 0.546 | 0.546 | 0.546 | 0.546 |
| Lysine | 0.487 | 0.512 | 0.512 | 0.487 | 0.512 | 0.512 | 0.487 | 0.512 | 0.512 |

### Example 1

### Implementation of Either Carbon Fixation Route Enables Production of P3HB Under Anaerobic Conditions from Glucose

It was calculated that P3HB cannot be made under anaerobic conditions from glucose as the sole carbon source using a wild-type E. coli metabolism and the previously disclosed P3HB synthesis pathway. Utilization of the 3HP Bi-Cycle enabled production of P3HB at a theoretical yield of 0.494 g/g under these conditions. Alternative inclusion of the 3HP/4HB cycle enabled P3HB production and increased the yield from 0.000 g/g to 0.618 g/g under anaerobic conditions. The overall stoichiometry (molar) of this pathway is reported in Equation E1.

1 Glucose→1.29P3HB+0.47CO₂+0.35 Formate+1.76 Water (Equation E1)

### Example 2

### Implementation of the Carbon Fixation Cycles Increases the Yield of P3HB from Glucose Under Aerobic Conditions

The maximum theoretical yield of P3HB from glucose was 0.478 g/g using the wild-type E. coli metabolism. Utilization of the 3HP Bi-Cycle increases the calculated theoretical yield to 0.589 g/g for cultivation on glucose under aerobic conditions. Alternatively, incorporation of the 3HP/4HB cycle increased the yield to 0.631 g/g, or an increase of 32% from the base E. coli metabolic network. The overall stoichiometry (molar) of this pathway is reported in Equation E2.

1 Glucose+0.056O2→1.32P3HB+0.72CO2+2.04 Water (Equation E2)

### Example 3

### Addition of Hydrogen as an Electron Donor does not Increase Yield of P3HB from Glucose Under Anaerobic Conditions

Addition of hydrogen as an available feed did not increase the yield of P3HB from glucose under anaerobic conditions. For the base metabolic network, hydrogen co-feeding with glucose was not sufficient to enable anaerobic production of P3HB. The yield remained 0.000 g/g. For the enhanced networks, the maximum theoretical yield remained constant at 0.618 g/g with the 3HP/4HB cycle and 0.494 g/g with the 3HP Bi-Cycle with or without hydrogen feeding.

### Example 4

### Addition of Hydrogen as an Electron Donor Increases Yield of P3HB from Glucose Under Aerobic Conditions

Addition of hydrogen as an electron donating substrate increase the yield to 0.717 with either of the carbon fixation routes implemented. This is compared to the initial yields of 0.589 g/g with the 3HP Bi-Cycle or 0.631 g/g with the 3HP/4HB cycle when hydrogen was not available. The overall stoichiometry (molar) of this new pathway is reported in Equation E4.

1 Glucose+3.5O₂+8.5H₂→1.5P3HB+10 Water (Equation E4)

### Example 5

### Addition of Hydrogen and CO₂ as Co-Feeds Increases Yield of P3HB from Glucose Under Aerobic Conditions when Carbon Fixation Cycles are Present

Under aerobic conditions and with either the heterologous 3HP/4HB cycle or 3HP Bi-Cycle and hydrogen co-feeding, the maximum theoretical yield of P3HB from glucose was 0.717 g/g as reported in Example 5 above. The addition of excess carbon dioxide as an available co-feed increased the yield to 0.956 g/g. The overall stoichiometry (molar) of this new pathway is reported in Equation E5.

1 Glucose+4.25O₂+14.5H₂+2CO₂→2.00P3HB+14.5 Water (Equation E5)

The addition of hydrogen and/or carbon dioxide co-feeds did not impact the maximum theoretical yield of the base case. This yield remained constant at 0.478 g/g.

### Example 6

### Implementation of Either Carbon Fixation Pathways Enables Production of P4HB From Glucose Under Anaerobic Conditions

It was calculated that P4HB cannot be synthesized from glucose under anaerobic conditions using the base E. coli metabolic network and previously reported synthesis routes. Incorporation of the 3HP/4HB cycle enabled production of P4HB anaerobically from glucose at a yield of 0.469 g/g. Alternative incorporation of the 3HP Bi-Cycle further increases the yield to 0.478 g/g.

### Example 7

### Additional Co-Feed of Hydrogen Increases Yield of P4HB from Glucose Under Anaerobic Conditions

It was calculated that P4HB cannot be synthesized from glucose under anaerobic conditions using the base E. coli metabolic network and previously reported P4HB synthesis routes. Addition of a hydrogen co-feed was sufficient to enable production of P4HB anaerobically from glucose at a yield of 0.478 g/g. The further addition of the 3HP/4HB cycle did not further enhance this yield, contrary to the observations in Example 6 above. The overall calculated stoichiometry is reported in Equation E7.

1 Glucose+1H₂→1P4HB+1 acetate+2 water (Equation E7)

### Example 8

### Implementation of the Carbon Fixation Cycles has No Effect on P4HB Production from Glucose Under Aerobic Conditions

Under aerobic conditions, the maximum theoretical yield of P4HB from glucose using the base E. coli metabolism was 0.597 g/g. This yield remains unchanged upon the incorporation of the 3HP/4HB cycle from Metallosphaera sedula or the 3HP Bi-Cycle.

### Example 9

### Additional Co-Feed of Hydrogen Increases Yield of P4HB from Glucose Under Aerobic Conditions

Under aerobic conditions, the maximum theoretical yield of P4HB from glucose using the base E. coli metabolism was 0.587 g/g. Upon addition of hydrogen as a co-feed, the yield increased to 0.717 g/g. This corresponded to a 22% increase in the maximum theoretical yield. Further incorporation of the 3HP/4HB Cycle or 3HP Bi-Cycle did not further increase this yield. The overall stoichiometry of this pathway is reported in Equation E9.

1 Glucose+7H₂+2.75O₂→1.5P4HB+8.5 water (Equation E9)

### Example 10

### Additional Co-Feeds of Hydrogen and CO₂ Increases Yield of P4HB from Glucose Under Aerobic Conditions

Under aerobic conditions, the maximum theoretical yield of P4HB from glucose using the base E. coli metabolism was 0.597 g/g. Upon addition of both hydrogen and carbon dioxide as co-feeds the yield of P4HB from glucose was increased to 0.956 g/g. This represented a 63% increase in the yield of P4HB from glucose, and a 33% increase in the yield when supplying hydrogen individually. Further incorporation of either carbon fixation cycle did not further improve this yield. The overall stoichiometry of this pathway is reported in Equation E10.

1 Glucose+16.25H2+5.125O2+2CO2→2P4HB+16.25 water (Equation E10)

### Example 11

### Incorporation of Carbon Fixation Pathways or Hydrogen Feeding does not Enable P5HV Synthesis Under Anaerobic Conditions

It was calculated that P5HV synthesis is not feasible utilizing the native E. coli metabolism with the previously described P5HV pathway under anaerobic cultivation with glucose as the sole carbon source. Unlike in P3HB and P4HB systems, neither carbon fixation pathway was able to enable P5HV synthesis under anaerobic cultivation on glucose. Also, implementation of hydrogen feeding or hydrogen plus carbon dioxide failed to enable P5HV synthesis under anaerobic conditions.

### Example 12

### Implementation of Both Carbon Fixation Pathways and Hydrogen Feeding Increases Yields of P5HV from Glucose Under Aerobic Conditions

The yield of P5HV from glucose was calculated as 0.437 g/g under aerobic cultivation on glucose. Implementation of hydrogen feeding increased this yield to 0.556 g/g, while further implementation of either carbon fixation pathway increased the theoretical yield to 0.667 g/g. This represented a 53% increase in the yield of P5HV from glucose.

### Example 13

### Feeding of Carbon Dioxide Increases Yield of P5HV from Glucose with Hydrogen Feeding and Carbon Fixation Pathways

The calculated yield of P5Hv from glucose on aerobic conditions with hydrogen and carbon dioxide feeding is 0.556 g/g, identical to the yield without carbon dioxide feeding. Implementation of either carbon dioxide fixation pathway increased the yield to 1.11 g/g on a glucose basis.

### Example 14

### Mixed Effects of the Carbon Fixation Cycles on the Yield of P3HP from Glucose

Under aerobic conditions, addition of the carbon fixation pathways had mixed effects on the yield of P3HP from glucose depending on the P3HP pathway considered. For the P3HP-mcr pathway, the yield increased from 0.649 g/g to 1.049 g/g when the 3HP Bi-cycle was included, but remained unchanged at 0.649 g/g when the 3HP/4HB recycle was considered. In the other routes, incorporation of a carbon fixation pathway resulted in small increases in the calculate yields from 0.667 g/g to 0.686 g/g for the P3HP-gol pathway and 0.615 g/g to 0.649 g/g for the P3HP-bAla pathway to P3HP.

### Example 15

### Mixed Effects of the Carbon Fixation Pathways on the Yield of P3HP from Glucose Under Anaerobic Conditions

The incorporation of carbon fixation pathways did not affect the yield of the P3HP-mcr pathway under anaerobic conditions. The yields calculated via the P3HP-gol and P3HP-bAla pathways increased when the 3HP/4HB cycle was included, raising the yields from 0.343 to 0.379 g/g for the P3HP-gol pathway and from 0.267 g/g to 0.313 g/g for the P3HP-bAla pathway. The yields remained unchanged from the base case for the 3HP Bi-Cycle pathway addition.

### Example 16

### Increased Yields for the Cultivation of P3HP from Glucose With Hydrogen Feeding Under Aerobic Conditions

Addition of hydrogen feeding increased yields under all combinations of metabolic pathways and P3HP synthesis routes. For most combinations, the resulting yield was 0.800 g/g representing a 16-30% increase against the same routes and carbon fixation pathway combinations when hydrogen was not available as a co-feed. For example, utilizing the P3HP-mcr route without hydrogen co-feed, the yield was calculated as 0.649 g/g. Inclusion of hydrogen co-feeds increased the yield to 0.80 g/g. For the P3HP-mcr route combined with the 3HP Bi-cycle, the feeding of hydrogen increased the yield from 1.05 g/g to 1.2 g/g

### Example 17

### The Yield of P3HP from Glucose is Increased Under Anaerobic Conditions when H₂ and CO₂ are Supplied in Combination with Carbon Fixation Pathways

The anaerobic cultivation of P3HP from glucose with hydrogen and carbon dioxide feeds resulted in no increase in calculated yields over the cases where glucose or glucose and hydrogen were used as the feeds. Incorporation of the 3HP/4HB cycle increase the yields to 0.436 g/g from 0.343 g/g (no cycle) or 0.379 (no carbon dioxide co-feeding) when utilizing the P3HP-gol pathway.

Utilization of the P3HP-mcr pathway in combination with glucose, hydrogen, and carbon dioxide co-feeding results in the largest improvement with a calculated yield of 0.733 g/g compared to the yield of 0.360 g/g with no carbon fixation pathway or 0.313 g/g with a more limited substrate set.

### Example 18

### Carbon Fixation Pathways Increase Yields of P3HP from Glucose Under Aerobic Conditions in Combination with Hydrogen and Carbon Dioxide Feeding

Further implementation of carbon dioxide feeding had no effect on the calculated yields from glucose compared to the hydrogen feeding case. In both cases, the calculated yield was 0.800 g/g. However, addition of carbon fixation routes increase most yields to 1.600 g/g. The exception is for the P3HP-mcr route combined with the 3HP Bi-Cycle which increases yield from 0.800 g/g to 2.00 g/g, or an increase of 150%.

### Example 19

### Mixed Effects of Carbon Fixation Pathways on the Yield of P3HB-4HB Copolymers from Glucose Under Anaerobic Conditions, while Hydrogen Feeds Alone have No Effect

For random P3HB-4HB copolymer containing 10% 4HB, addition of the carbon fixation pathways increased yields of polymer from glucose under anaerobic conditions. In the base case, the yield was calculated to be 0.368 g/g. Addition of carbon fixation pathways increased the yield to 0.531 g/g and 0.604 g/g for the 3HP Bi-Cycle and 3HP/4HB cycles, respectively. For other copolymer compositions (50% 4HB and 90% 4HB), no effect was calculated.

Hydrogen feeding was no observed to effect these calculations, with the yields remaining constant across all compositions and pathway options. Further addition of carbon dioxide co-feeding also did not further increase yields.

### Example 20

### Mixed Effect of Carbon Fixation Pathways on the Yield of P3HB-4HB Copolymers from Glucose Under Aerobic Conditions, with Increased Yields Associated with Hydrogen Feeding

Similar to the previous example, addition of the 3HP Bi-Cycle increase the yields of P3HB-10%-4HB from 0.503 g/g to 0.597 g/g while the use of the 3HP/4HB cycle increase the yield to 0.630 g/g. The remaining compositions (50% and 90% 4HB) resulted in no increase in yields under aerobic glucose utilization.

Unlike the previous example, the utilization of a hydrogen co-feed increased the yields of random copolymer across most calculations. While the production of P3HB-10%-4HB remained flat at 0.503 g/g with the addition of hydrogen co-feed, the yields of P3HB-50%-4HB increased from 0.630 to 0.637 g/g and the yield of P3HB-90%-4HB increased from 0.601 to 0.717 g/g due to hydrogen feeding in the base metabolism.

Further augmentation of the metabolic network with carbon fixation cycles increased the calculated yields of all copolymer compositions to 0.717 g/g.

### Example 21

### CO₂ feeding increases yield of P3HB-4HB copolymers from glucose with hydrogen feeding and carbon fixation pathways

For all compositions of P3HB-4HB random copolymers, the yield of copolymer from glucose increased from 0.717 g/g to 0.956 g/g when excess carbon dioxide was available as substrate in the cases where carbon fixation pathways have been used to augment the E. coli metabolic network and hydrogen and glucose are consumed in an aerobic environment.

### Example 22

### Carbon Fixation Pathways and/or Hydrogen Feeding is Insufficient to Enable the Production of P3HB-5HV Random Copolymers From Glucose Under Anaerobic Conditions

It was calculated across all P3HB-5HV copolymer compositions that the production of copolymer from glucose under anaerobic conditions is infeasible. The addition of carbon dioxide fixation pathways or hydrogen feeding, or both, did not enable copolymer synthesis under anaerobic conditions.

### Example 23

### Mixed Results for the Effects of Carbon Fixation Pathways on the Yield of P3HB-5HV from Glucose while Hydrogen Feeding Improves Yields in Systems with Active Carbon Fixation Pathways

The yield of P3HB-10%-5HV was calculated to improve upon the addition of either the 3HP Bi-Cycle or the 3HP/4HB cycle under aerobic conditions. The 3HP Bi-Cycle increased the yield of this composition from 0.486 g/g to 0.573 g/g while the 3HP/4HB cycle increased the yield to 0.604 g/g. Other compositions were unaffected by the addition of the carbon fixation pathways to the metabolic network.

The addition of hydrogen consumption to the base metabolic network had no effect on the yield of 10% or 50% 5HV copolymer but did increase the yield of P3HB-90%-5HV from 0.451 g/g to 0.548 g/g. In systems containing carbon fixation pathways, incremental addition of hydrogen increased the yields to 0.711, 0.689, and 0.671 g/g for 10%, 50%, and 90% 5HV copolymer. This represents an 18% to 49% improvement in the yield of copolymer from glucose.

The incremental addition of carbon dioxide as a co-feed had no effect on yield.

### Example 24

### Combined Utilization of Carbon Fixation Pathways and Hydrogen Feeds Increase Yields of 1,4-Butanediol from Glucose

Under aerobic conditions, the maximum theoretical yield of BDO from glucose was calculated as 0.546 g/g. Incremental additions of carbon fixation pathways were insufficient to increase this yield.

Addition of hydrogen feeding to the base case increased the yield to 0.750 g/g. Again, incremental addition of the carbon fixation pathways did not increase this yield.

Addition of the carbon dioxide as substrate further increased the yield to 1.00 g/g. Incorporation of the 3HP Bi-Cycle increased this yield to 2.00 g/g while the 3HP/4HB cycle had no effect.

Under anaerobic conditions, it was calculated that the maximum theoretical yield of BDO from glucose was 0.546 g/g. This calculated yield was unchanged with the addition of carbon dioxide fixation pathway, hydrogen feeding, and carbon dioxide feeding.

### Example 25

### Contributions of Hydrogen Feeding and Carbon Dioxide Fixation Pathways to the Yields of 1,3-Propanediol from Glucose

Under aerobic conditions, the yield of PDO was calculated as 0.618 g/g using glucose as the sole substrate. Addition of either carbon dioxide fixation route to the metabolic network increased the yield to 0.633 g/g.

Addition of hydrogen feeding to the base aerobic case increased the yield from 0.618 g/g to 0.844 g/g. Incremental addition of either carbon dioxide fixation route did not further increase yields. Combining either carbon fixation route with both hydrogen and carbon dioxide feeding under aerobic conditions increased the yields to 1.689 g/g, a two-fold increase over the hydrogen utilizing cases and a 173% increase over the base case.

Under anaerobic conditions, utilization of carbon fixation pathways increased the theoretical yield from 0.563 g/g under the base case to 0.633 g/g with either carbon fixation pathway. Further incremental adjustment of the system to include either hydrogen feeding with or without carbon dioxide feeding did not result in an increase in the calculated theoretical yields.

### Example 26

### Mixed Observations on the Effect of Carbon Fixation Pathways On the Production of 3HP from Glucose Under Anaerobic Conditions

Under anaerobic conditions, it was calculated that the utilization of either carbon dioxide fixation pathway increased the yield from 0.600 g/g to 0.818 g/g when 3HP is synthesized via the 3HP-bAla route. A smaller effect is observed in the 3HP-gol route, where the yield is increased from 0.600 g/g to 0.692 g/g. No effect is observed for the 3HP-mer route.

While no effect is observed under anaerobic conditions upon the incremental addition of hydrogen feeding, the addition of both hydrogen and carbon dioxide feeding results in yield improvement. For the 3HP-mcr route, the yield is increased from 0.818 to 0.857 g/g with no further increase observed from the addition of carbon fixation pathways. For the 3HP-gol route, the yield is increased from 0.692 to 0.750 g/g when either carbon fixation route is present. The yield of 3HP from glucose improves in the 3HP-bAla route from 0.818 to 0.857 g/g upon the addition of hydrogen and carbon dioxide feeding when a carbon fixation route is present.

### Example 27

### Incremental Increases to the Yield of 3HP from Glucose Under Aerobic Conditions Upon the Utilization of Hydrogen Feeding and Carbon Fixation Pathways

Under aerobic conditions, a small improvement on the calculated maximum yield of 3HP from glucose was observed when carbon fixation pathways were supplemented to the base metabolic network. For 3HP-got, the yield increased from 0.909 to 0.938 g/g while the yield increased from 0.909 to 0.968 g/g for the 3HP-bAla pathway. No improvement was calculated in the addition of either carbon dioxide fixation pathway to the 3HP-mcr synthesis route.

Addition of hydrogen feeding to the system increased all yields to 1.00 g/g regardless of the supplementation of the metabolic network with carbon fixation pathways. Further incorporation of carbon dioxide excess did not improve the base case, the yields of pathways supplemented with carbon fixation routes increased to 2.00 g/g.

### Example 28

### Contributions of Hydrogen Feeding and Carbon Dioxide Fixation Pathways to the Yields of Lysine from Glucose

Under aerobic conditions, the calculated lysine yield from glucose was 0.640 g/g. The addition of either carbon fixation pathway increased this yield to 0.649. A larger increase to 0.811 g/g was calculated utilizing the base metabolic network combined with hydrogen consumption. Incremental addition of carbon fixation pathways did not increase this yield. However, when carbon fixation pathways were combined with carbon dioxide over-supply, the simulated yields increased from 0.811 g/g to 1.622 g/g, or an increase of 100%.

Under anaerobic conditions, the yield of lysine from glucose was calculated as 0.487 g/g. Addition of either carbon fixation pathway increased this yield to 0.512 g/g. Further modifications of the system (hydrogen feeding with or without excess carbon dioxide) did not further impact these anaerobic yields.

### Example 29

### Utilization of Hydrogen and Carbon Dioxide Feeding Increases Maximum Yields for Production of 1,5-Pentanediol from Glucose Under Aerobic Cultivation, Especially with Expression of Carbon Fixation Pathways

Under aerobic conditions, the calculated 1,5-pentanediol (15PDO) yield from glucose was 0.432 g/g. The addition of either carbon fixation pathway did not increase this yield. A yield increase to 0.578 g/g was calculated upon the addition of hydrogen feeding or hydrogen feeding with excess carbon dioxide. A further increase to 0.693 g/g (hydrogen feeding) or 1.16 g/g (hydrogen and carbon dioxide feeding) was calculated with the further addition of either carbon fixation pathway.

### Example 30

### Utilization of Hydrogen and Carbon Dioxide Feeding Increases Maximum Yields for the Production of Glutarate from Glucose Under Aerobic Cultivation, Especially with Expression of Carbon Fixation Pathways

Under aerobic conditions, the calculated glutarate yield from glucose was 0.698 g/g. The addition of either carbon fixation pathway did not increase this yield. A yield increase to 0.733 was calculated upon the addition of hydrogen feeding. Further addition of excess carbon dioxide did not increase this yield further.

The calculated yield increased to 0.880 g/g with the addition of either carbon fixation pathway with hydrogen feeding. A further increase to 1.467 g/g was calculated with the combination of hydrogen and carbon dioxide feeding with expression of either carbon fixation pathway.

### Example 31

### Utilization of Hydrogen and Carbon Dioxide Feeding Increases Maximum Yields for the Production of Delta-Valerolactone from Glucose Under Aerobic Cultivation, Especially with Expression of Carbon Fixation Pathways

Under aerobic conditions, the calculated delta-valerolactone yield from glucose was 0.437 g/g. The addition of either carbon fixation pathway did not significantly increase this yield.

A yield increase to 0.556 g/g was observed with the addition of either hydrogen feeding or hydrogen feeding with carbon dioxide. Further yield increase to 0.667 g/g was calculated for the case when hydrogen feeding is combined with either of the carbon fixation cycles. In the case where hydrogen and carbon dioxide feeding were combined with the carbon fixation cycles, the calculated yield increased to 1.111 g/g.

As described in Example 28 and shown in Table 3, the pathways described herein create lysine under both anaerobic and anaerobic conditions. Under aerobic conditions and in the presence of glucose, the calculated yield of lysine was 0.649 g/g, which increased to 0.811 g/g utilizing the base metabolic network combined with hydrogen consumption. Additionally, carbon fixation pathways combined with carbon dioxide and hydrogen increased the calculated yield to 1.622 g/g.

Accordingly, it is possible to further modify the synthetic pathways disclosed herein to include a lysine pathway to produce glutarate, 5-hydroxyvalerate, poly-5-hydroxyvalerate (P5HV), delta-valerolactone, and 1,5-pentanediol, as shown in FIG. 3. As disclosed in WO 2010/068953, which is incorporated herein by reference in its entirety, an exemplary host can express one or more genes encoding lysine 2-monooxygenase, 5-aminopentanamidase, 5-aminopentanoate transaminase, glutarate semialdehyde reductase, 5-hydroxyvalerate CoA-transferase, and polyhydroxyalkanoate synthase to produce a PHA polymer containing 5HV monomers. Preferably the host has deletions or mutations in genes encoding glutarate semialdehyde dehydrogenase and/or lysine exporter encoding genes. Particularly suitable hosts also have the ability to overproduce lysine and are resistant to toxic analogs, like S-(2-aminoethyl) cysteine.

Other than in the examples herein, or unless otherwise expressly specified, all of the numerical ranges, amounts, values and percentages, such as those for amounts of materials, elemental contents, times and temperatures of reaction, ratios of amounts, and others, in the following portion of the specification and attached claims may be read as if prefaced by the word “about” even though the term “about” may not expressly appear with the value, amount, or range. Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains error necessarily resulting from the standard deviation found in its underlying respective testing measurements. Furthermore, when numerical ranges are set forth herein, these ranges are inclusive of the recited range end points (i.e., end points may be used). When percentages by weight are used herein, the numerical values reported are relative to the total weight.

Also, it should be understood that any numerical range recited herein is intended to include all sub-ranges subsumed therein. For example, a range of “1 to 10” is intended to include all sub-ranges between (and including) the recited minimum value of 1 and the recited maximum value of 10, that is, having a minimum value equal to or greater than 1 and a maximum value of equal to or less than 10. The terms “one,” “a,” or “an” as used herein are intended to include “at least one” or “one or more,” unless otherwise indicated.

The teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

What is claimed is:

1. A method of producing a polyhydroxyalkanoate (PHA) monomer, polymer or co-polymer, comprising the steps of:
a) growing a genetically engineered organism having a carbon fixation pathway and a PHA pathway in the presence of a carbon feedstock, wherein the yield of the PHA monomer, polymer, co-polymer, or combinations thereof is greater than the yield produced by a wild-type organism homologous having either a carbon fixation pathway or a PHA pathway.

2. The method of claim 1, wherein the carbon fixation pathway is a 3HP/4HB cycle pathway or a 3HP bi-cycle pathway.

3. The method of claim 2, wherein the monomer is 3-hydroxyproprionate, 3-hydroxybutyrate, 4-hydroxybutyrate, or 5-hydroxyvalerate.

4. The method of claim 3, wherein the polymer is poly-3-hydroxypropionate, poly-3-hydroxybutyrate, poly-4-hydroxybutyrate, poly-5-hydroxyvalerate, or copolymers thereof.

5. The method of any one of claim 1, wherein the monomer is further enzymatically processed to a diol.

6. The method of claim 5, wherein the monomer is 3-hydroxypropionate and the diol is 1,3-propanediol.

7. The method of claim 5, wherein the monomer is 4-hydroxybutyrate and the diol is 1,4 butanediol.

8. The method of claim 5, wherein the monomer is 5-hydroxyvalerate and the diol is 1,5-petanediol.

9. The method of claim 1, wherein the PHA pathway is a poly-3-hydroxypropionate pathway.

10. The method of claim 1, wherein the organism is grown in aerobic conditions.

11. The method of claim 1, wherein the organism is grown in anaerobic conditions.

12. The method of claim 1, wherein the growth conditions further include a hydrogen co-feed.

13. The method of claim 1, wherein the growth conditions further include a carbon dioxide co-feed.

14. The method of claim 1, wherein the growth conditions further include a hydrogen co-feed and a carbon dioxide co-feed.

15. The method of claim 1, wherein the organism is selected from Escherichia coli, Ralstonia eutropha (Cupravidus necator, Alcaligenes eutrophus, Metallosphaera sedula, Sulfolobus genus, Pyrobaculum genus, Caldivirga maquilingensis, Thermoproteus neutrophilus, Acinetobacter baumannii, Acinetobacter baylyi, Acinetobacter aceti, Acinetobacter sp. DR1, Acinetobacter calcoaceticus, Acinetobacter haemolyticus, Acinetobacter johnsonii, Acinetobacter junii, Acinetobacter lwoffii, Acinetobacter radioresistens, Acinetobacter venetianus, Acinetobacter sp. DSM, Zoogloea ramigera, Allochromatium vinosum, Rhodococcus ruber, Delftia acidovorans, Aeromonas caviae, Synechocystis sp. PCC 6803, Synechococcus elongatus PCC 7942, Thiocapsa pfenigii, Bacillus megaterium, Clostridium kluyveri, Methylobacterium extorquens, Nocardia corralina, Nocardia salmonicolor, Pseudomonas fluorescens, Pseudomonas oleovorans, Pseudomonas sp. 6-19, Pseudomonas sp.61-3 and Pseudomonas putida, Rhodobacter sphaeroides, Alcaligenes latus, Klebsiella oxytoca, Anaerobiospirillum succiniciproducens, Actinobacillus succinogenes, Mannheimia succiniciproducens, Rhizobium etli, Bacillus subtilis, Corynebacterium glutamicum, Gluconobacter oxydans, Zymomonas mobilis, Lactococcus lactis, Lactobacillus plantarum, Streptomyces coelicolor, Clostridium acetobutylicum, Saccharomyces cerevisiae, Schizosaccharomyces pombe, Kluyveromyces lactis, Kluyveromyces marxianus, Aspergillus terreus, Aspergillus niger, Pichia pastoris, Chlorella spp., Chlorella minutissima, Chlorella emersonii, Chlorella sorokiniana, Chlorella ellipsoidea, Chlorella sp., and Chlorella protothecoides.

16. The method of claim 1, wherein the organism has a genetic modification in at least one gene coding for an enzyme selected from the group consisting of: (1) Acetyl-CoA carboxylase; (2) malonyl-CoA reductase; (3) propionyl-CoA synthase; (4) propionyl-CoA carboxylase; (5) methylmalonyl-CoA epimerase; (6) methylmalonyl-CoA mutase; (7) succinyl-CoA:(S)-malate-CoA transferase; (8) succinate dehydrogenase; (9) fumarate hydratase; (10 a, b, c) (S)-malyl-CoA/β-methylmalyl-CoA/(S)-citramalyl-CoA (MMC) lyase; (11) mesaconyl-C1-CoA hydratase (β-methylmalyl-CoA dehydratase); (12) mesaconyl-CoA C1-C4 CoA transferase; and (13) mesaconyl-C4-CoA hydratase.

17. The method of claim 16, wherein the genes coding for at least two of the enzymes are genetically modified.

18. The method of claim 16, wherein the genes coding for at least three of the enzymes are genetically modified.

19. The method of claim 1, wherein the organism has a genetic modification in at least one gene coding for an enzyme selected from the group consisting of: (1) acetyl-CoA carboxylase; (2) malonyl-CoA reductase (NADPH); (3) malonate semialdehyde reductase (NADPH); (4) 3-hydroxypropionyl-CoA synthetase (AMP-forming); (5) 3-hydroxypropionyl-CoA dehydratase; (6) acryloyl-CoA reductase (NADPH); (7) propionyl-CoA carboxylase; (8) methylmalonyl-CoA epimerase; (9) methylmalonyl-CoA mutase; (10) succinyl-CoA reductase (NADPH); (11) succinate semialdehyde reductase (NADPH); (12) 4-hydroxybutyryl-CoA synthetase (AMP-forming); (13) 4-hydroxybutyryl-CoA dehydratase; (14) crotonyl-CoA hydratase; (15) 3-hydroxybutyryl-CoA dehydrogenase (NAD+); and (16) acetoacetyl-CoA β-ketothiolase.

20. The method of claim 19, wherein the genes coding for at least two of the enzymes are genetically modified.

21. The method of claim 19, wherein the genes coding for at least three of the enzymes are genetically modified.

22. The method of claim 1, wherein the organism is E. coli, the PHA pathway is poly-3-hydroxybutyrate (P3HB), and the organism is grown under anaerobic conditions.

23. The method of claim 22, wherein the growth conditions further include a hydrogen co-feed or a carbon dioxide co-feed.

24. The method of claim 22, wherein the growth conditions further include a hydrogen co-feed and a carbon dioxide co-feed.

25. The method of claim 1, wherein in organism is E. coli, the PHA pathway is a poly-3-hydroxybutyrate (P3HB), and the organism is grown under aerobic conditions.

26. The method of claim 25, wherein the growth conditions further include a hydrogen co-feed or a carbon dioxide co-feed.

27. The method of claim 25, wherein the growth conditions further include a hydrogen co-feed and a carbon dioxide co-feed.

28. The method of claim 1, wherein the organism is E. coli, the PHA pathway is poly-4-hydroxybutyrate, and the organism is grown under anaerobic conditions.

29. The method of claim 28, wherein the growth conditions further include a hydrogen co-feed or a carbon dioxide co-feed.

30. The method of claim 28, wherein the growth conditions further include a hydrogen co-feed and a carbon dioxide co-feed.

31. The method of claim 1, wherein the organism is E. coli, the PHA pathway is poly-4-hydroxybutyrate, and the organism is grown under aerobic conditions.

32. The method of claim 31, wherein the growth conditions further include a hydrogen co-feed or a carbon dioxide co-feed.

33. The method of claim 31, wherein the growth conditions further include a hydrogen co-feed and a carbon dioxide co-feed.

34. The method of claim 1, wherein the organism is E. coli, the PHA pathway is poly-5-hydroxyvalerate, and the organism is grown under anaerobic conditions.

35. The method of claim 34, wherein the growth conditions further include a hydrogen co-feed or a carbon dioxide co-feed.

36. The method of claim 34, wherein the growth conditions further include a hydrogen co-feed and a carbon dioxide co-feed.

37. The method of claim 1, wherein the organism is E. coli, the PHA pathway is poly-5-hydroxyvalerate, and the organism is grown under aerobic conditions.

38. The method of claim 37, wherein the growth conditions further include a hydrogen co-feed or a carbon dioxide co-feed.

39. The method of claim 37, wherein the growth conditions further include a hydrogen co-feed and a carbon dioxide co-feed.

40. The method of claim 1, wherein the organism is E. coli, the PHA pathway is poly-3-hydroxypropionoate, and the organism is grown under anaerobic conditions.

41. The method of claim 40, wherein the growth conditions further include a hydrogen co-feed or a carbon dioxide co-feed.

42. The method of claim 40, wherein the growth conditions further include a hydrogen co-feed and a carbon dioxide co-feed.

43. The method of claim 40, wherein the PHA pathway proceeds via a substrate comprising malonyl-coA, glycerol, and beta-alanine.

44. The method of claim 1, wherein the organism is E. coli, the PHA pathway is poly-3-hydroxypropionoate, and the organism is grown under aerobic conditions.

45. The method of claim 43, wherein the growth conditions further include a hydrogen co-feed or a carbon dioxide co-feed.

46. The method of claim 43, wherein the growth conditions further include a hydrogen co-feed and a carbon dioxide co-feed.

47. The method of claim 43, wherein the PHA pathway proceeds via a substrate comprising malonyl-coA, glycerol, and beta-alanine.

48. The method of claim 1, wherein the organism is E. coli, the PHA pathway is a poly-3-hydroxybutyrate-co-4-hydroxybutyrate polymer pathway, and the organism is grown under aerobic conditions.

49. The method of claim 48, wherein the growth conditions further include a hydrogen co-feed or a carbon dioxide co-feed.

50. The method of claim 48, wherein the growth conditions further include a hydrogen co-feed and a carbon dioxide co-feed.

51. The method of claim 1, wherein the organism is E. coli, the PHA pathway produces 1,4-butanediol, and the organism is grown under aerobic conditions with a hydrogen co-feed or a carbon dioxide co-feed.

52. The method of claim 1, wherein the organism is E. coli, the PHA pathway produces 1,3-propanediol, and the organism is grown under aerobic conditions with a hydrogen co-feed or a carbon dioxide co-feed.

53. The method of claim 1, wherein the organism is E. coli, the PHA pathway produces 1,5-pentanediol product, and the organism is grown under aerobic conditions with a hydrogen co-feed or a carbon dioxide co-feed.

54. The method of claim 1, wherein the organism further includes a genetically incorporated hydrogenase gene or the method includes upregulating a hydrogenase gene.

55. The method of claim 1, wherein the carbon feedstock is glucose.

56. The method of claim 1, wherein the carbon feedstock is sucrose or a sugar derived from a cellulosic hydrolysate.

57. An organism selected from the group consisting of:
a) an organism homologously having a carbon fixation pathway, wherein the organism is genetically engineered to incorporate a polyhydroxyalkanoate pathway for producing a polyhydroxyalkanoate monomer, polymer or copolymer;
b) an organism homologously capable of producing a polyhydroxyalkanoate polymer, wherein the organism is genetically engineered to incorporate a carbon fixation pathway; and
c) an organism genetically engineered to incorporate a polyhydroxyalkanoate pathway and a carbon fixation pathway.

58. The organism of claim 57, wherein the carbon fixation pathway is capable of utilizing glucose, sucrose, or a sugar derived from a cellulosic hydrolysate as a carbon source.

59. The organism of claim 57, wherein the organism is further genetically engineered to incorporate a lysine pathway for producing lysine.

60. The organism of claim 59, wherein the organism provides increased yield of lysine compared to the organism before incorporation of the carbon fixation pathway.

61. The organism of claim 60, wherein the incorporated lysine pathway produces poly-5-hydroxyvalerate, 5-hydroxyvalerate, glutarate, δ-valeralactone, or 1,5-pentanediol.

62. The organism of claim 61, wherein the organism is grown in the presence of a hydrogen or carbon dioxide feed.

63. The organism of claim 61, wherein the organism expresses express one or more genes encoding lysine 2-monooxygenase, 5-aminopentanamidase, 5-aminopentanoate transaminase, glutarate semialdehyde reductase, 5-hydroxyvalerate CoA-transferase, and polyhydroxyalkanoate synthase.

64. A method for producing a diol, comprising the steps of:
a) providing an organism capable of producing diol;
b) genetically engineering the organism by incorporating a carbon fixation pathway to convert glucose to acetyl-CoA when the organism is grown in the presence of glucose as a carbon source, thereby producing a diol-producing organism genetically engineered to utilize glucose; and
c) providing glucose to the diol-producing organism genetically engineered to utilize glucose.

65. A method for producing a diol, comprising the steps of:
a) providing an organism;
b) genetically engineering the organism by incorporating a carbon fixation pathway to convert glucose to acetyl-CoA when the organism is grown in the grown in the presence of glucose;
c) genetically engineering the organism by incorporating a diol pathway, thereby producing an organism genetically engineered to utilize glucose to produce a diol; and
d) providing glucose to the organism genetically engineered to utilize glucose to produce a diol.


SEQUENCE LISTING (18 sequences)

SEQ ID NO: 1 - DNA, 35 bp, Artificial Sequence
  Unknown
ttgacagctagctcagtcctaggtataatgctagc

SEQ ID NO: 2 - DNA, 35 bp, Artificial Sequence
  Unknown
ttgacagctagctcagtcctaggtactgtgctagc

SEQ ID NO: 3 - DNA, 35 bp, Artificial Sequence
  Unknown
tttacagctagctcagtcctaggtattatgctagc

SEQ ID NO: 4 - DNA, 35 bp, Artificial Sequence
  Unknown
ctgacagctagctcagtcctaggtataatgctagc

SEQ ID NO: 5 - DNA, 35 bp, Artificial Sequence
  Unknown
tttacggctagctcagtcctaggtacaatgctagc

SEQ ID NO: 6 - DNA, 35 bp, Artificial Sequence
  Unknown
ttgacagctagctcagtcctagggactatgctagc

SEQ ID NO: 7 - DNA, 30 bp, Artificial Sequence
  Unknown
ttgacaattaatcatccggctcgtataatg

SEQ ID NO: 8 - DNA, 33 bp, Artificial Sequence
  Unknown
ttgacaattaatcatcgtcgtataatgtgtgga

SEQ ID NO: 9 - DNA, 54 bp, Artificial Sequence
  Unknown
tccctatcagtgatagagattgacatccctatcagtgatagagatactgagcac

SEQ ID NO: 10 - DNA, 31 bp, Artificial Sequence
  Unknown
tcgccagtctggcctgaacatgatataaaat

SEQ ID NO: 11 - DNA, 178 bp, Artificial Sequence
  Unknown
aaccactatcaatatattcatgtcgaaaatttgtttatctaacgagtaagcaaggcggattgacggatcatccgggtcgctataaggtaaggatggtcttaacactgaatccttacggctgggttagccccgcgcacgtagttcgcaggacgcgggtgacgtaacggcacaagaaacg

SEQ ID NO: 12 - DNA, 170 bp, Artificial Sequence
  Unknown
atgcgggttgatgtaaaactttgttcgcccctggagaaagcctcgtgtatactcctcacccttataaaagtccctttcaaaaaaggccgcggtgctttacaaagcagcagcaattgcagtaaaattccgcaccattttgaaataagctggcgttgatgccagcggcaaac

SEQ ID NO: 13 - DNA, 35 bp, Artificial Sequence
  Unknown
ttgacagctagctcagtcctaggtacagtgctagc

SEQ ID NO: 14 - DNA, 35 bp, Artificial Sequence
  Unknown
ttgacagctagctcagtcctaggtacaatgctagc

SEQ ID NO: 15 - DNA, 29 bp, Artificial Sequence
  Unknown
ctaatgagcgggcttttttttgaacaaaa

SEQ ID NO: 16 - DNA, 38 bp, Artificial Sequence
  Unknown
aaaaaaaaaaaaccccgcttcggcggggtttttttttt

SEQ ID NO: 17 - DNA, 44 bp, Artificial Sequence
  Unknown
ataaaacgaaaggctcagtcgaaagactgggcctttcgttttat

SEQ ID NO: 18 - DNA, 28 bp, Artificial Sequence
  Unknown
agaaggccatcctgacggatggcctttt